(19) United States
(12) Patent Application Publication
(10) Pub. No.: US 2008/0125377 A1
(43) Pub. Date: May 29, 2008

(54) DELIVERY SYSTEM FOR ANTIANGIOGENIC AND ANTIINFLAMMATORY PHARMACEUTICALS AND METHOD OF USE

(76) Inventors: Stephen P. Bartels, Pittsford, NY (US); Tim Tak Lam, Arcadia, CA (US); Afshin Shafiee, Rochester, NY (US); Yongqing Lin, Rochester, NY (US)

Correspondence Address: Bausch & Lomb Incorporated, One Bausch & Lomb Place, Rochester, NY, 14604-2701, US

(21) Appl. No.: 11/563,719

(22) Filed: Nov. 28, 2006

(51) Int. Cl. A61K 31/505

(52) U.S. Cl. 514/23; 514/272

(57) ABSTRACT

Diseases and conditions associated with tissues of the body, including but not limited to tissues in the eye, can be effectively treated, prevented, inhibited, onset delayed, or regression caused by administering therapeutic agents to those tissues. Described herein are formulations which deliver a variety of therapeutic agents, including but not limited to BOL-303213-X, to a subject for an extended period of time. The formulation may be placed in an aqueous medium of a subject, including but not limited to via intraocular or periocular administration, or placement proximate to a site of a disease or condition to be treated in a subject. A method may be used to administer BOL-303213-X to treat or prevent angiogenesis, choroidal neovascularization, or age-related macular degeneration, or wet age-related macular degeneration in a subject. The formulations may comprise BOL-303213-X or other therapeutic agents.

### CROSS-REFERENCES TO RELATED APPLICATIONS

None

### FIELD

Described herein are formulations for treatment, prevention, inhibition, delaying onset of, or causing regression of an angiogenic- or inflammatory-mediated disease or condition of the posterior segment by delivery of therapeutic agents to a subject, or a formulation comprising a therapeutic agent, to the eye of a subject.

### BACKGROUND

There is a variety of angiogenic- or inflammatory-mediated retinal disorders for which there is currently no treatment or for which the current treatment is not optimal. Retinal disorders such as uveitis (an inflammation of the uveal tract: iris, ciliary body, and choroid), central retinal vein occlusive diseases (CRVO), branch retinal venous occlusion (BRVO), macular degeneration, macular edema, diabetic macular edema, proliferative diabetic retinopathy, and retinal detachment generally are all retinal disorders that are difficult to treat with conventional therapies. These retinal disorders, as well as other disorders of the posterior chamber, are mediated by angiogenic and/or inflammatory mechanisms.

Age-related macular degeneration (AMD) is the major cause of severe visual loss in the United States for individuals over the age of 60. AMD occurs in either an atrophic or less commonly an exudative form. The atrophic form of AMD is also called “dry AMD,” and the exudative form of AMD is also called “wet AMD.”

In exudative AMD, blood vessels grow from the choriocapularis through defects in Bruch's membrane, and in some cases the overlying retinal pigment epithelium. Organization of serous or hemorrhagic exudates escaping from these vessels results in fibrovascular scarring of the macular region with attendant degeneration of the neuroretina, detachment and tears of the retinal pigment epithelium, vitreous hemorrhage and permanent loss of central vision. This process is responsible for more than 80% of cases of significant visual loss in subjects with AMD. Current or forthcoming treatments include laser photocoagulation, photodynamic therapy, treatment with VEGF antibody fragments, treatment with pegylated aptamers, and treatment with certain small molecule agents.

Choroidal neovascularization (CNV) has proven to be recalcitrant to treatment in most cases. Conventional laser treatment can ablate CNV and help to preserve vision in selected cases not involving the center of the retina, but this is limited to only about 10% of the cases. Unfortunately, even with successful conventional laser photocoagulation, the neovascularization recurs in about 50-70% of eyes (50% over 3 years and >60% at 5 years). (Macular Photocoagulation Study Group, Arch. Ophthalmol. 204:694-701 (1986)). In addition, many subjects who develop CNV are not good candidates for laser therapy because the CNV is too large for laser treatment, or the location cannot be determined so that the physician cannot accurately aim the laser. Photodynamic therapy, although utilized in up to 50% of new cases of subfoveal CNV has only marginal benefits over natural history, and generally delays progression of visual loss rather than improving vision which is already decreased secondary to the subfoveal lesion. PDT is neither preventive nor definitive. Several PDT treatments are usually required per subject and additionally, certain subtypes of CNV fare less well than others.

Thus, there remains a long-felt need for methods, compositions, and formulations that may be used to optimally prevent or significantly inhibit angiogenic- or inflammatory-mediated posterior chamber diseases such as choroidal neovascularization and to prevent and treat wet AMD.

### SUMMARY

Described herein are methods, compositions and formulations for administering to a human subject an amount of BOL-303213-X effective to treat, prevent, inhibit, delay onset of, or cause regression of wet AMD. BOL-303213-X is a fused pyrrolocarbazole which inhibits the human tyrosine kinase with Ig and epidermal growth factor homology domain-2 (TIE-2) and human vascular endothelial growth factor receptor-2 (VEGF-R2) tyrosine kinases. These two receptor kinases are known to play important roles in stabilization of blood vessels and angiogenesis.

As described in further detail in the Detailed Description section, the methods, compositions and formulations may also be used for delivery to a subject, including but not limited to a human subject or to the eye of a human subject of therapeutically effective amounts of BOL-303213-X for the treatment, prevention, inhibition, delaying of the onset of, or causing the regression of wet AMD. In some variations, the methods, compositions, and liquid formulations are used to treat wet AMD. In some variations, the methods, compositions, and liquid formulations are used to prevent wet AMD. In some variations, the methods and formulations described herein are used to prevent the transition from dry AMD to wet AMD. The methods, compositions and formulations may also be used for delivery to a subject, including but not limited to a human subject or to the eye of a subject therapeutically effective amounts of BOL-303213-X for the treatment, prevention, inhibition, delaying of the onset of, or causing the regression of choroidal neovascularization (CNV). In some variations, the methods, compositions and liquid formulations are used to treat CNV. The methods, compositions and formulations may also be used for delivery to a subject, including but not limited to a human subject or to the eye of a subject, of therapeutically effective amounts of BOL-303213-X for the treatment, prevention, inhibition, delaying of the onset of, or causing the regression of angiogenesis in the eye. In some variations, the methods, compositions and formulations are used to treat angiogenesis. Other diseases and conditions that may be treated, prevented, inhibited, have onset delayed, or caused to regress using BOL-303213-X are described in the Diseases and Conditions section of the Detailed Description.

As described in further detail in the Detailed Description, the methods, compositions and formulations may also be used for delivery to a subject, including but not limited to a human subject or to the eye of a subject therapeutically effective amounts of therapeutic agents other than BOL-303213-X for the treatment, prevention, inhibition, delaying of the onset of, or causing the regression of wet AMD. In some variations, the methods, compositions and liquid formulations are used to treat wet AMD. Therapeutic agents that may be used are described in detail in the Therapeutic Agents section. Such therapeutic agents include but are not limited to dual pan-VEGFR and TIE-2 inhibitor compounds. Dual pan-VEGFR and TIE-2 inhibitor compounds that may be used include but are not limited to the fused pyrrolocarbazole family of compounds described further in the Therapeutic Agents section herein, including BOL-303213-X, derivatives, analogs, prodrugs, salts and esters thereof. The methods, compositions and formulations may also be used for delivery to a subject, including but not limited to a human subject or to the eye of a subject of therapeutically effective amounts of anti-angiogenic or anti-inflammatory therapeutic agents for the treatment, prevention, inhibition, delaying of the onset of, or causing the regression of CNV. In some variations, the methods, compositions and formulations are used to treat CNV. The methods, compositions and formulations may also be used for delivery to a subject, including but not limited to a human subject or to the eye of a subject of therapeutically effective amounts of therapeutic agents for the treatment, prevention, inhibition, delaying of the onset of, or causing the regression of angiogenesis-mediated in the eye disease. In some variations, the methods, compositions and formulations are used to treat angiogenesis-mediated disease of the posterior chamber. The methods, compositions and formulations may also be used for delivery to a subject, including but not limited to a human subject or to the eye of a subject of therapeutically effective amounts of therapeutic agents for the treatment, prevention, inhibition, delaying of the onset of, or causing the regression of inflammatory-mediated disease in the eye. In some variations, the methods, compositions and formulations are used to treat inflammatory-mediated disease of the posterior chamber. Other diseases and conditions that may be treated, prevented, inhibited, have onset delayed, or caused to regress using therapeutic agents other than BOL-303213-X are described in the Diseases and Conditions section of the Detailed

### DESCRIPTION

One formulation described herein comprises a therapeutic agent in a solvent. Generally, any solvent that has the desired effect may be used in which the therapeutic agent dissolves and/or suspends and which can be administered to a subject, including but not limited to a human subject or an eye of a subject. Generally, any concentration of therapeutic agent that has the desired effect can be used. The formulation in some variations is a solution which is unsaturated, a saturated or a supersaturated solution. The solvent may be a pure solvent or may be a mixture of liquid solvent components. In some variations the solution formed is an in situ gelling formulation. Solvents and types of solutions that may be used are well known to those versed in such drug delivery technologies.

The formulations described herein may form a dispersed or non-dispersed mass when placed into a rabbit eye, including but not limited to the vitreous of a rabbit eye. In some variations the non-dispersed mass comprises a gel. In some variations, the liquid formulation comprises a therapeutic agent and a plurality of polymers. In some variations one of the polymers is polycaprolactone. In some variations one of the polymers is PEG 400.

In some variations, the non-dispersed mass comprises a depot. In some variations, the non-dispersed mass consists of a drug delivery device.

For liquid formulations which form a non-dispersed mass, the non-dispersed mass may generally be any geometry or shape. The non-dispersed mass-forming liquid formulations may, for instance, appear as a compact spherical mass when placed in the vitreous. In some variations the liquid formulations described herein form a milky or whitish colored semi-contiguous or semi-solid non-dispersed mass relative to the medium in which it is placed, when placed in the vitreous.

The liquid formulations may generally be administered in any volume that has the desired effect. In one method a volume of a liquid formulation is administered to the vitreous and the liquid formulation is less than 200 μl.

Routes of administration that may be used to administer a liquid formulation include but are not limited to placement of the liquid formulation by placement, including by injection, into a medium, including but not limited to an aqueous medium in the body, including but not limited to intraocular or periocular injection. In some variations, the liquid formulation is administered subconjunctivally, periconjunctivally or retrobulbarly. In some variations, the liquid formulation is administered to the sub-Tenon. In some variations, the liquid formulation is administered intravitreally.

The liquid formulations described herein may be delivered to any medium of a subject, including but not limited to a human subject, including but not limited to an aqueous medium of a subject.

One liquid formulation described herein comprises a liquid formulation of BOL-303213-X or other therapeutic agent. The liquid formulations may comprise a solution, suspension, an in situ gelling formulation, or an emulsion. The droplets in the emulsion may generally be of any size, including but not limited to up to about 5,000 nm.

In some formulations described herein, the formulations may comprise a therapeutic agent including but not limited to BOL-303213-X, and one or more solubilizing agents or solvents. In some variations, the solubilizing agent or solvent is glycerin, DMSO, DMA, N-methylpyrrolidone, ethanol, benzyl alcohol, benzyl benzoate, isopropyl alcohol, polyethylene glycol of various molecular weights, including but not limited to PEG 300 and PEG 400, propylene glycol, propylene carbonate or water or a mixture of one or more thereof.

The liquid formulations described herein may deliver a therapeutic agent or agents for an extended period of time. One nonlimiting example of such an extended release delivery system is a liquid formulation that delivers a therapeutic agent or agents to a subject, including but not limited to a human subject or to the eye of a subject in an amount sufficient to maintain an amount effective to treat, prevent, inhibit, delay onset of, or cause regression of a disease or condition in a subject for an extended period of time. In some variations, the liquid formulation is used to treat a disease or condition in a subject, including but not limited to a human subject. In some variations, the liquid formulation delivers the therapeutic agent for at least about one, about two, about three, about six, about nine, about twelve months, about 18 months, or about 24 months.

The formulations described herein may deliver BOL-303213-X or other therapeutic agents for an extended period of time. One nonlimiting example of such an extended release delivery system is a liquid formulation that delivers BOL-303213-X to a subject, including but not limited to a human subject or to the eye of a subject in an amount sufficient to maintain an amount effective to treat, prevent, inhibit, delay onset of, or cause regression of wet age-related macular degeneration for an extended period of time. In some variations, the formulation is used to treat wet age-related macular degeneration for an extended period of time. In some variations, the formulation is used to prevent wet age-related macular degeneration for an extended period of time. In some variations, the liquid formulation is used to prevent transition of dry AMD to wet AMD for an extended period of time. In one nonlimiting example, the formulation delivers the BOL-303213-X to the vitreous, sclera, retina, choroid, macula, or other tissues of a subject, including but not limited to a human subject in an amount sufficient to treat, prevent, inhibit, delay onset of, or cause regression of wet age-related macular degeneration for at least about three, about six, about nine, about twelve months, about 18 months, or about 24 months. In some variations, the level of BOL-303213-X is sufficient to treat AMD. In some variations, the level of BOL-303213-X is sufficient to prevent onset of wet AMD.

Other extended periods of release are described in the Detailed Description.

### BRIEF DESCRIPTION OF THE DRAWINGS

FIG. 1 is a graphical representation of the effect of BOL-303213-X on rhVEGF165 induced vascular leakage;

FIG. 2 is a graphical representation of the effect of BOL-303213-X on percent of Grade IV lesions in cynomolgus monkeys.

### DETAILED DESCRIPTION

Described herein are compositions, formulations and methods relating to delivery of therapeutic agents to a subject, including but not limited to a human subject or to the eye of a subject. These compositions, formulations, and methods may be used for the treatment, prevention, inhibition, delaying onset of, or causing regression of angiogenic- or inflammatory-mediated diseases and conditions of the eye including but not limited to diseases or conditions of the posterior chamber, including but not limited to choroidal neovascularization; macular degeneration; diabetic macular edema; diabetic retinopathy; age-related macular degeneration, including wet AMD and dry AMD; retinal angiogenesis; chronic uveitis; and other retinoproliferative conditions. In some variations, the compositions, formulations, and methods are used for the treatment of the aforementioned diseases or conditions of the eye.

Herein are described (1) therapeutic agents that may be delivered to a subject, including, but not limited to a, human subject or an eye of a subject using the compositions, liquid formulations, and methods described herein, (2) diseases and conditions that may be treated, prevented, inhibited, onset delayed, or regression caused by delivery of the therapeutic agents, (3) liquid formulations that may be used to deliver the therapeutic agents, (4) routes of administration for delivery of the liquid formulations, (5) extended delivery of therapeutic agents including but not limited to BOL-303213-X, and (6) description of the treatment of CNV and wet AMD by delivery of BOL-303213-X to a subject, including but not limited to a human subject or to the eye of a subject for an extended period of time using the described compositions and liquid formulations.

The term “about,” as used herein, refers to the level of accuracy that is obtained when the methods described herein, such as the methods in the examples, are used. However, by “about” a certain amount of a component of a formulation is meant 90-110% of the amount stated.

### Therapeutic Agents

Most generally, any compounds and compositions currently known or yet to be discovered that are useful in treating, preventing, inhibiting, delaying the onset of, or causing the regression of the diseases and conditions described herein may be therapeutic agents for use in the compositions, liquid formulations, and methods described herein.

The fused pyrrolocarbazole family of compounds may be used in the compositions, liquid formulations and methods for the treatment, prevention, inhibition, delaying the onset of, or causing the regression of angiogenesis- and inflammatory-mediated diseases and conditions of the eye, including diseases set forth in the disease and indication section of this application. BOL-303213-X and BOL-303213-X derivatives and analogs may be used to prevent, treat, inhibit, delay the onset of, or cause regression of angiogenesis-mediated diseases and conditions of the eye, including choroidal neovascularization. BOL-303213-X may be used to prevent, treat, inhibit, delay the onset of, or cause regression of AMD, including wet AMD. In some variations, a member of the fused pyrrolocarbazole family of compounds or BOL-303213-X is used to treat wet AMD or angiogenesis-mediated diseases and conditions of the eye including choroidal neovascularization.

BOL-303213-X is a member of a family of compounds described in US Patent Appln. Pub. No. 2005/0143442 the contents of which are incorporated by reference herein.

BOL-303123-X is represented by the following structural formula:

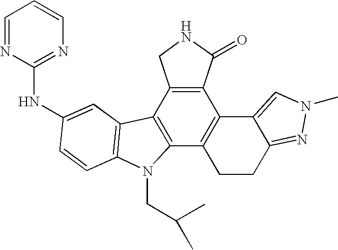

Other compounds in the BOL-303213-X family of compounds include those having the following structural formula:

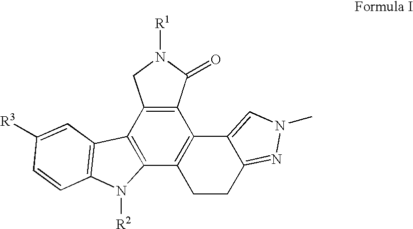

wherein R¹ is selected from H, substituted alkyl, and unsubstituted alkyl, R² is H, optionally substituted alkyl, optionally substituted alkenyl, optionally substituted alkynyl, or optionally substituted cycloalkyl, and R³ is selected from at least one of the following paragraphs.

1. OR¹³; especially those where R¹³ is optionally substituted cycloalkyl, and particularly those where the cycloalkyl is a 5 or 6 membered ring.

2. C(═O)R¹³; especially those where R¹³ is optionally substituted cycloalkyl, and particularly those where the cycloalkyl is a 5 or 6 membered ring.

3. (alkylene)OR¹³; especially those where R¹³ is optionally substituted cycloalkyl, and particularly those where the cycloalkyl is a 5 or 6 membered ring.

4. (CH₂)ₚOR²²; especially those where R²² is a branched chain alkyl.

5. O-(alkylene)-R²⁷.

6. OCH(CO₂R¹⁸)₂; especially those where R¹⁸ is optionally substituted alkyl.

7. OCH[(CH₂)ₚOR²⁰]₂; especially those where R²⁰ is optionally substituted alkyl.

8. C(═O)-(alkylene)-R²⁵.

9. NR¹¹R³³; especially those where R³³ is optionally substituted heteroaryl.

10. (alkylene)-NR¹⁸R¹⁹; especially those where R¹⁸ is H or optionally substituted alkyl.

11. C(R¹²)═N—R¹⁸; especially those where R¹² is alkyl, and those where R¹⁸ is optionally substituted heterocycloalkyl.

12. CH═N—OR¹³; especially those where R¹³ is optionally substituted heterocycloalkyl.

13. C(R¹²)═N—OR²⁰; especially those where R¹² and R³⁰ are optionally substituted alkyl.

14. C(R¹¹)═N—NR¹¹C(═O)NR^{14A}R^{14B}.

15. C(R¹¹)═N—NR¹¹SO₂R¹³.

16. OC(═O)NR¹¹(alkylene)-R²⁶; especially those where R²⁶ is optionally substituted aryl or optionally substituted heteroaryl.

17. OC(═O)[N(CH₂CH.₂)₂N]—R²¹; especially those where R²¹ is optionally substituted heteroaryl.

18. NR¹¹C(═O)OR²³; especially those where R²³ is optionally substituted aryl.

19. NR¹¹C(═O)S—R¹⁸.

20. NR¹¹C(═—)NR¹¹R²³; especially those where R²³ is optionally substituted aryl or optionally substituted heteroaryl.

21. NR¹¹C(═S)NR¹¹R²³; especially those where R²³ is optionally substituted aryl.

22. NR¹¹S(═O)₂N(R¹⁵)₂.

23. NR¹¹C(═O)NR¹¹(alkylene)-R²⁴; especially those where R²⁴ is optionally substituted heterocycloalkyl, or optionally substituted heteroaryl.

24. NR¹¹C(═O)N(R¹¹)NR^{16A}R^{16B}.

25. substituted alkyl, wherein one of the substituents is an optionally substituted spirocycloalkyl group.

26. optionally substituted (alkylene)ₓ-cycloalkyl, especially optionally substituted (C₁-alkylene)-cycloalkyl and optionally substituted cycloalkyl.

27. optionally substituted-(alkylene)ₓ-heterocycloalkyl, wherein the heterocycloalkyl does not include unsubstituted N-morpholinyl, N-piperidyl, or N-thiomorpholinyl; especially optionally substituted (C₁-alkylene)-heterocycloalkyl, optionally substituted heterocycloalkyl, and more especially optionally substituted heterocycloalkyl groups with two heteroatoms, optionally substituted tetrahydrofuranyl and optionally substituted tetrahydropyranyl.

28. NR.¹¹R³², especially those where R³² is a phenyl group and wherein the phenyl group is optionally substituted with one or more alkoxy groups, and in particular, with one or more methoxy groups.

As used herein, the term “alkyl” refers to a straight-chain, or branched alkyl group having 1 to 8 carbon atoms, such as methyl, ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, octyl, etc. The alkyl moiety of alkyl-containing groups, such as alkoxy, alkoxycarbonyl, and alkylaminocarbonyl groups, has the same meaning as alkyl defined above. Lower alkyl groups, which are preferred, are alkyl groups as defined above which contain 1 to 4 carbons. A designation such as “C₁-C₄ alkyl” refers to an alkyl radical containing from 1 to 4 carbon atoms.

As used herein, the term “alkenyl” refers to a straight chain, or branched hydrocarbon chains of 2 to 8 carbon atoms having at least one carbon-carbon double bond. A designation “C₂-C₈ alkenyl” refers to an alkenyl radical containing from 2 to 8 carbon atoms. Examples of alkenyl groups include ethenyl, propenyl, isopropenyl, 2,4-pentadienyl, etc.

As used herein, the term “alkynyl” refers to a straight chain, or branched hydrocarbon chains of 2 to 8 carbon atoms having at least one carbon-carbon triple bond. A designation “C₂-C₈ alkynyl” refers to an alkynyl radical containing from 2 to 8 carbon atoms. Examples include ethynyl, propynyl, isopropynyl, 3,5-hexadiynyl, etc.

As used herein, the term “alkylene” refers to a branched or straight chained hydrocarbon of 1 to 8 carbon atoms, which is formed by the removal of two hydrogen atoms. A designation such as “C₁-C₄ alkylene” refers to an alkylene radical containing from 1 to 4 carbon atoms. Examples include methylene (—CH₂—), propylidene (CH₃CH₂CH═), 1,2-ethandiyl (—CH₂CH₂—), etc.

As used herein, the term “cycloalkyl” refers to a saturated or partially saturated mono- or bicyclic alkyl ring system containing 3 to 10 carbon atoms. A designation such as “C₅-C₇ cycloalkyl” refers to a cycloalkyl radical containing from 5 to 7 ring carbon atoms. Preferred cycloalkyl groups include those containing 5 or 6 ring carbon atoms. Examples of cycloalkyl groups include cyclopropyl, cyclobutyl, cyclopentyl, cyclohexyl, cyclohexenyl, cycloheptyl, cyclooctyl, etc.

As used herein, the term “heterocycloalkyl” refers to a cycloalkyl group in which one or more ring carbon atoms are replaced by at least one hetero atom such as —O—, —N—, or —S—, and includes ring systems which contain a saturated ring group bridged or fused to one or more aromatic groups. Some heterocycloalkyl groups containing both saturated and aromatic rings include phthalamide, phthalic anhydride, indoline, isoindoline, tetrahydroisoquinoline, chroman, isochroman, and chromene.

As used herein, the term “spirocycloalkyl” refers to a cycloalkyl group bonded to a carbon chain or carbon ring moiety by a carbon atom common to the cycloalkyl group and the carbon chain or carbon ring moiety. For example, a C3 alkyl group substituted with an R group wherein the R group is spirocycloalkyl containing 5 carbon atoms refers to:

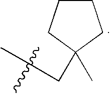

As used herein, the term “heteroaryl” refers to an aryl group containing 5 to 10 ring carbon atoms in which one or more ring carbon atoms are replaced by at least one hetero atom such as —O—, —N—, or —S—. Some heteroaryl groups of the present invention include pyridyl, pyrimidyl, pyrrolyl, furanyl, thienyl, imidazolyl, triazolyl, tetrazolyl, quinolyl, isoquinolyl, benzoimidazolyl, thiazolyl, pyrazolyl, and benzothiazolyl groups.

Other therapeutic agents that may be used include anti-inflammatory agents, including, but not limited to nonsteroidal anti-inflammatory agents and steroidal anti-inflammatory agents. In some variations, active agents that may be used in the liquid formulations are ACE-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, antibacterials, antihypertensives, pressors, antiprotozoal agents, antiviral agents, antifungal agents, anti-infective agents, antitumor agents, antimetabolites, and antiangiogenic agents.

In some variations, cortisone, dexamethasone, fluocinolone, hydrocortisone, loteprednol, methylprednisolone, prednisolone, prednisone, dissociated glucocorticoid receptor agonists (DIGRA compounds) and triamcinolone, or their salts, esters or other derivatives, may be used. The liquid formulation may include a combination of two or more steroidal therapeutic agents.

In some variations the formulation comprises a combination of one or more therapeutic agents.

### Diseases and Conditions

Diseases and conditions that may be treated, prevented, inhibited, onset delayed, or regression caused herein are the described diseases and conditions that may be treated, prevented, inhibited, onset delayed, or regression caused using the therapeutic agents and the formulations, liquid formulations, and methods described herein. In some variations, the diseases or conditions are treated using the therapeutic agents and the formulations, liquid formulations, and methods described herein. Unless the context indicates otherwise, it is envisioned that the subjects on whom all of the methods of treatment may be performed include, but are not limited to, human subjects.

Generally, any diseases or condition of the eye susceptible to treatment, prevention, inhibition, delaying the onset of, or causing the regression of using the therapeutic agents and the formulations, liquid formulations and methods described herein may be treated, prevented, inhibited, onset delayed, or regression caused treated or prevented. Examples of diseases or conditions of the eye include, but are not limited to, diseases or conditions associated with angiogenesis- or inflammatory-mediated diseases or conditions including retinal and/or choroidal neovascularization.

Angiogenisis- or inflammatory-mediated diseases or conditions associated the eye that can be treated, prevented, inhibited, have onset delayed, or be caused to regress using the formulations, liquid formulations, and methods described herein include, but are not limited to, ocular diseases/pathologies such as melanoma, alkali burns, pteragtium, herpetic stromal keratitis, trachoma, proliferative diabetic retinopathy, retinopathy of prematurity, choroidal and retinal neovascularization, ocular melanoma, glaucoma etc. Many of these diseases/pathologies also involve inflammation. In addition, many ocular diseases/pathologies such as diabetic edema, macular edema, dry eye, allergic conjunctivitis, retinal degeneration, glaucoma, etc. involve inflammation with typical vascular changes. The concept underlying the current invention is to take advantage of the dual actions of BOL-303123-X and apply it to ocular diseases that involve angiogenesis or inflammation or both. This application can be used alone or together with other therapies (surgical and/or medical) to treat ocular diseases involving angiogenesis and/or inflammation and/or tissue remodeling such as diabetic retinopathy including proliferative diabetic retinopathy, macular degeneration, wet and dry AMD, retinopathy of prematurity (retrolental fibroplasia), infections causing a retinitis or choroiditis, presumed ocular histoplasmosis, myopic degeneration, angioid streaks, and ocular trauma. Other non-limiting examples of diseases and conditions of the eye that may be treated, prevented, inhibited, have onset delayed, or be caused to regress using the formulations, liquid formulations, and methods described herein include, but are not limited to, pseudoxanthoma elasticum, vein occlusion, artery occlusion, carotid obstructive disease, Sickle Cell anemia, Eales disease, myopia, chronic retinal detachment, hyperviscosity syndromes, toxoplasmosis, trauma, polypoidal choroidal vasculopathy, post-laser complications, complications of idiopathic central serous chorioretinopathy, complications of choroidal inflammatory conditions, rubeosis, diseases associated with rubeosis (neovascularization of the angle), neovascular glaucoma, uveitis and chronic uveitis, macular edema including diabetic macular edema, proliferative retinopathies and diseases or conditions caused by the abnormal proliferation of fibrovascular or fibrous tissue, including all forms of proliferative vitreoretinopathy (including post-operative proliferative vitreoretinopathy), whether or not associated with diabetes.

In some variations, the formulations and pharmaceutical formulations described herein are used to prevent or delay onset of a angiogenic- or inflammatory-mediated disease or condition of the eye where the subject, including but not limited to a human subject, is at heightened risk of developing the disease or condition of the eye. A subject with a heightened risk of developing a disease or condition is a subject with one or more indications that the disease or condition is likely to develop in the particular subject. In some variations the subject with a heightened risk of developing wet AMD is a subject with dry AMD in at least one eye. In some variations the subject with a heightened risk of developing wet AMD in a fellow eye is a subject with wet AMD in the other eye. In some variations, the formulations and pharmaceutical formulations described herein are used to prevent or delay onset of CNV in a subject at heightened risk of developing CNV, including but not limited to prevention or delaying onset of CNV in the fellow eye of a subject, including but not limited to a human subject with AMD in one eye. In some variations, the formulations and pharmaceutical formulations described herein are used to prevent or delay onset of CNV in the fellow eye of a subject with wet AMD in one eye. In some variations, the formulations and pharmaceutical formulations comprise a dual pan-VEGFR and TIE-2 inhibitor compound, including but not limited to BOL-303213-X. In some variations the formulations and pharmaceutical formulations are administered periocularly, including without limitation subconjunctivally, to a human subject with vision of 20/40 or better. In some variations, the formulations and pharmaceutical formulations are administered periocularly, including without limitation subconjunctivally, to the eye of a human subject where the eye to which the formulation is administered has vision of 20/40 or better.

In some variations, the formulations and pharmaceutical formulations described herein are used to treat, prevent, or delay onset of AMD. In some variations, the formulations and pharmaceutical formulations described herein are used to treat, prevent, or delay onset of dry AMD. In some variations, subjects including but not limited to human subjects with non-central geographic atrophy are administered a formulation or pharmaceutical formulations described herein to treat, prevent, or delay onset of central geographic atrophy. In some variations, the formulations and pharmaceutical formulations comprise a fused pyrrolocarbazole compound, including but not limited to BOL-303213-X. In some variations the formulations and pharmaceutical formulations are administered periocularly, including without limitation subconjunctivally, periconjunctivally or retrobulbarly to a human subject with vision of 20/40 or better. In some variations, the formulations and pharmaceutical formulations described herein are administered and the subject, including but not limited to a human subject is also treated with a second therapy for treating the disease or disorder. The second therapy can comprise surgical and/or medical therapies to treat ocular diseases involving angiogenesis, inflammation and/or tissue remodeling. In some variations, the formulations and pharmaceutical formulations described herein are used to treat, prevent, or delay onset of wet or dry AMD and the subject, including but not limited to a human subject is also treated with laser therapy such as photodynamic laser therapy, either before, during, or after treatment with the formulations or pharmaceutical formulations described herein.

One disease that may be treated, prevented, inhibited, have onset delayed, or be caused to regress using the formulation, liquid formulations and methods described herein is the wet form of AMD. In some variations wet AMD is treated using the formulations, liquid formulations and methods described herein. The wet form of AMD is characterized by blood vessels growing from their normal location in the choroid into an undesirable position under the retina. Leakage and bleeding from these new blood vessels results in vision loss and possibly blindness.

The formulations, liquid formulations, and methods described herein may also be used to prevent or slow the transition from the dry form of AMD (wherein yellow deposits called drusen form under the retinal pigment epithelium (RPE) causing the RPE to degenerate and leading to photoreceptor cell death) to the wet form of AMD.

“Macular degeneration” is characterized by the excessive buildup of fibrous deposits in the macula and retina and the atrophy of the retinal pigment epithelium. As used herein, an eye “afflicted” with macular degeneration is understood to mean that the eye exhibits at least one detectable physical characteristic associated with the disease of macular degeneration. The administration of BOL-303213-X appears to limit and regress angiogenesis, such as choroidal neovascularization in age-related macular degeneration (AMD), which may occur without treatment. As used herein, the term “angiogenesis” means the generation of new blood vessels (“neovascularization”) into a tissue or organ. An “angiogenesis-mediated disease or condition” of the eye or retina is one in which new blood vessels are generated in a pathogenic manner in the eye or retina, resulting in diminution or loss of vision or other problem, e.g., choroidal neovascularization associated with AMD.

An “inflammatory-mediated disease or condition” of the eye or retina is one in which a localized protective response is elicited by injury or destruction of tissues, which serves to destroy, dilute, or wall off (sequester) both the injurious agent and the injured tissue. It should be understood that not every disease or condition that may be considered to be an inflammatory-mediated disease or condition will present all of the symptoms or characteristics associated with inflammation. For example, DME is not a protective response but rather is a degenerative response due to retinal microangiopathy. In the acute form, inflammation is characterized by the classical signs of pain, heat, redness, swelling, and loss of function. DME is not characterized by pain, heat or redness. Histologically, inflammation involves a complex series of events, including dilation of arterioles, capillaries, and venules, with increased permeability and blood flow; exudation of fluids, including plasma proteins; and leukocyte migration into the inflammatory focus. DME is not characterized by dilation of arterioles, capillaries and venules. DME is not characterized by leukocyte migration into the edematous area. Chronic inflammation is inflammation of slow progress and marked chiefly by the formation of new connective tissue. It may be a continuation of an acute form and usually causes permanent tissue damage. DME is not characterized by formation of new connective tissue in a pathogenic manner in the eye or retina, resulting in diminution or loss of vision or other problem, e.g., choroidal neovascularization associated with AMD.

As used herein, to “inhibit” a disease or condition by administration of a therapeutic agent means that the progress of at least one detectable physical or functional characteristic or symptom of the disease or condition is slowed or stopped following administration of the therapeutic agent as compared to the progress of the disease or condition without administration of the therapeutic agent.

As used herein, to “prevent” a disease or condition by administration of a therapeutic agent means that the detectable physical characteristics or symptom of the disease or condition do not develop following administration of the therapeutic agent.

As used herein, to “delay onset of” a disease or condition by administration of a therapeutic agent means that at least one detectable physical characteristic or symptom of the disease or condition develops later in time following administration of the therapeutic agent as compared to the progress of the disease or condition without administration of the therapeutic agent.

As used herein, to “treat” a disease or condition by administration of a therapeutic agent means that the progress of at least one detectable physical characteristic or symptom of the disease or condition is slowed, stopped, or reversed following administration of the therapeutic agent as compared to the progress of the disease or condition without administration of the therapeutic agent.

As used herein, to “cause regression of” a disease or condition by administration of a therapeutic agent means that the progress of at least one detectable physical characteristic or symptom of the disease or condition is reversed to some extent following administration of the therapeutic agent.

A subject, including but not limited to a human subject, having a predisposition for or in need of prevention may be identified by the skilled practitioner by established methods and criteria in the field given the teachings herein. The skilled practitioner may also readily diagnose individuals as in need of inhibition or treatment based upon established criteria in the field for identifying angiogenesis and/or neovascularization and/or inflammation given the teachings herein.

As used herein, a “subject” is generally any animal that may benefit from administration of the therapeutic agents described herein. In some variations the therapeutic agents are administered to a mammalian subject. In some variations the therapeutic agents are administered to a human subject. In some variations the therapeutic agents may be administered to a veterinary animal subject. In some variations the therapeutic agents may be administered to a model experimental animal subject.

### Liquid Formulations

The liquid formulations described herein contain a therapeutic agent and may generally be any liquid formulation, including but not limited to solutions, suspensions, and emulsions. In some variations the liquid formulations form a non-dispersed mass relative to a surrounding medium when placed in, for example, the vitreous of a rabbit eye.

When a certain volume of a liquid formulation is administered, it is understood that there is some imprecision in the accuracy of various devices that may be used to administer the liquid formulation. Where a certain volume is specified, it is understood that this is the target volume. However, certain devices such as insulin syringes are inaccurate to greater than 10%, and sometimes inaccurate to greater than 20% or more. Hamilton HPLC type syringes are generally considered precise to within 10%, and are recommended when volumes below 10 μl are to be injected.

In some variations, a volume of a liquid formulation described herein is administered to the vitreous of a rabbit eye or a subject's, including but not limited to a human subject's eye that is less than about 200 μl, less than about 100 μl, less than about 90 μl, less than about 80 μl, less than about 70 μl, less than about 60 μl, less than about 50 μl, less than about 40 μl, less than about 30 μl, less than about 20 μl, less than about 10 μl, less than about 5 μl, less than about 3 μl, or less than about 1 μl. In some variations, a volume of a liquid formulation described herein is administered to the vitreous of a rabbit eye or subject's, including but not limited to a human subject's eye that is less than about 20 μl. In some variations, a volume of a liquid formulation described herein is administered to the vitreous that is less than about 10 μl. In some variations, a volume of a liquid formulation described herein is administered to the vitreous of a rabbit eye or a subject's, including but not limited to a human subject's eye that is between about 0.1 μl and about 200 μl, between about 50 μl and about 175 μl, between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some variations, a volume of a liquid formulation described herein is administered to the vitreous of a rabbit eye or a subject's, including but not limited to a human subject's eye that is between about 1 μl and about 10 μl. In some variations, a volume of a liquid formulation described herein is administered to the vitreous of a rabbit eye or a subject's, including but not limited to a human subject's eye that is between about 1 μl and about 5 μl. In some variations, a volume of a liquid formulation described herein is administered to the vitreous of a rabbit eye or a subject's eye that is between about 1 μl and about 5 μl. In some variations, a volume of a liquid formulation described herein is administered to the vitreous of a rabbit eye or a subject's, including but not limited to a human subject's eye that is between about 0.1 μl and about 200 μl.

In some variations, a total volume of a liquid formulation described herein is periocularly administered to a rabbit eye or a subject's, including but not limited to a human subject's eye that is less than about 1000 μl, less than about 900 μl less than about 800 μl, less than about 700 μl, less than about 600 μl, less than about 500 μl, less than about 400 μl, less than about 300 μl, less than about 200 μl less than about 100 μl. less than about 90 μl less than about 80 μl, less than about 70 μl less than about 60 μl, less than about 50 μl, less than about 40 μl, less than about 30 μl, less than about 20 μl, less than about 10 μl, less than about 5 μl, less than about 3 μl, or less than about 1 μl. In some variations, a volume of a liquid formulation described herein is periocularly administered to a rabbit eye or a subject's, including but not limited to a human subject's eye that is less than about 20 μl. In some variations, a volume of a liquid formulation described herein is periocularly administered to a rabbit eye or a subject's, including but not limited to a human subject's eye that is less than about 10 μl. In some variations, a volume of a liquid formulation described herein is periocularly administered to a rabbit eye or a subject's, including but not limited to a human subject's eye that is between about 0.1 μl and about 200 μl, between about 50 μl and about 200 μl, between about 200 μl and about 300 μl, between about 300 μl and about 400 μl, between about 400 μl and about 500 μl, between about 600 μL and about 700 μl, between about 700 μl and about 800 μl, between about 800 μL and about 900 μl, between about 900 μl and about 1000 μl between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some variations, a volume of a liquid formulation described herein is periocularly administered to a rabbit eye or a subject's, including but not limited to a human subject's eye that is between about 1 μl and about 10 μl. In some variations, a volume of a liquid formulation described herein is periocularly administered to a rabbit eye or a subject's, including but not limited to a human subject's eye that is between about 1 μl and about 5 μl. In some variations, a volume of a liquid formulation described herein is periocularly administered to a rabbit eye or a subject's, including but not limited to a human subject's eye that is between about 1 μl and about 5 μl. In some variations, a volume of a liquid formulation described herein is periocularly administered to a rabbit eye or a subject's, including but not limited to a human subject's eye that is between about 0.1 μl and about 200 μl.

In some variations, a total volume of a liquid formulation described herein is administered to the subconjunctival region of a rabbit eye or a subject's, including but not limited to a human subject's eye that is less than about 1000 μl, less than about 900 μl less than about 800 μl, less than about 700 μl, less than about 600 μl, less than about 500 μl, less than about 400 μl. less than about 300 μl, less than about 200 μl less than about 100 μl. less than about 90 μl less than about 80 μl, less than about 70 μl less than about 60 μl, less than about 50 μl, less than about 40 μl, less than about 30 μl, less than about 20 μl, less than about 10 μl, less than about 5 μl, less than about 3 μl, or less than about 1 μl. In some variations, a volume of a liquid formulation described herein is administered to the subconjunctival region of a rabbit eye or a subject's, including but not limited to a human subject's eye that is less than about 20 μl. In some variations, a volume of a liquid formulation described herein is administered to the subconjunctival region of a rabbit eye or a subject's, including but not limited to a human subject's eye that is less than about 10 μl. In some variations, a volume of a liquid formulation described herein is administered to the subconjunctival region of a rabbit eye or a subject's, including but not limited to a human subject's eye that is between about 0.1 μl and about 200 μl, between about 50 μl and about 200 μl, between about 200 μl and about 300 μl, between about 300 μl and about 400 μl, between about 400 μl and about 500 μl, between about 600 μL and about 700 μl, between about 700 μl and about 800 μl, between about 800 μL and about 900 μl, between about 900 μl and about 1000 μl between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some variations, a volume of a liquid formulation described herein is administered to the subconjunctival region of a rabbit eye or a subject's, including but not limited to a human subject's eye that is between about 1 μl and about 10 μl. In some variations, a volume of a liquid formulation described herein is administered to the subconjunctival region of a rabbit eye or a subject's, including but not limited to a human subject's eye that is between about 1 μl and about 5 μl. In some variations, a volume of a liquid formulation described herein is administered to the subconjunctival region of a rabbit eye or a subject's, including but not limited to a human subject's eye that is between about 1 μl and about 5 μl. In some variations, a volume of a liquid formulation described herein is administered to the subconjunctival region of a rabbit eye or a subject's, including but not limited to a human subject's eye that is between about 0.1 μl and about 200 μl.

In some variations, a total volume of a liquid formulation described herein is administered to the sub-Tenon region of a rabbit eye or a subject's, including but not limited to a human subject's eye that is less than about 1000 μl, less than about 900 μl less than about 800 μl, less than about 700 μl, less than about 600 μl, less than about 500 μl, less than about 400 μl. less than about 300 μl, less than about 200 μl less than about 100 μl. less than about 90 μl less than about 80 μl, less than about 70 μl less than about 60 μl, less than about 50 μl, less than about 40 μl, less than about 30 μl, less than about 20 μl, less than about 10 μl, less than about 5 μl, less than about 3 μl, or less than about 1 μl. In some variations, a volume of a liquid formulation described herein is administered to the sub-Tenon region of a rabbit eye or a subject's, including but not limited to a human subject's eye that is less than about 20 μl. In some variations, a volume of a liquid formulation described herein is administered to the sub-Tenon region of a rabbit eye or a subject's, including but not limited to a human subject's eye that is less than about 10 μl. In some variations, a volume of a liquid formulation described herein is administered to the sub-Tenon region of a rabbit eye or a subject's, including but not limited to a human subject's eye that is between about 0.1 μl and about 200 μl, between about 50 μl and about 200 μl, between about 200 μl and about 300 μl, between about 300 μl and about 400 μl, between about 400 μl and about 500 μl, between about 600 μL and about 700 μl, between about 700 μl and about 800 μl, between about 800 μL and about 900 μl, between about 900 μl and about 1000 μl between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some variations, a volume of a liquid formulation described herein is administered to the sub-Tenon region of a rabbit eye or a subject's, including but not limited to a human subject's eye that is between about 1 μl and about 10 μl. In some variations, a volume of a liquid formulation described herein is administered to the sub-Tenon region of a rabbit eye or a subject's, including but not limited to a human subject's eye that is between about 1 μl and about 5 μl. In some variations, a volume of a liquid formulation described herein is administered to the sub-Tenon region of a rabbit eye or a subject's, including but not limited to a human subject's eye that is between about 1 μl and about 5 μl. In some variations, a volume of a liquid formulation described herein is administered to the sub-Tenon region of a rabbit eye or a subject's, including but not limited to a human subject's eye that is between about 0.1 μl and about 200 μl.

In some variations the liquid formulations described herein are administered in multiple periocular locations within a period of time, including without limitation within an hour of one another. Without being bound by theory, it is thought that such multiple administrations, such as multiple injections, allow for a greater total dose to be administered periocularly than a single dose due to a potentially limited ability of the local ocular tissues to absorb larger volumes.

In some variations of the liquid formulations described herein, the therapeutic agent is a solution or suspension of BOL-303213-X in a liquid medium. Liquid media include but are not limited to solvents, including but not limited to those in the Solubilization of Therapeutic Agents section.

The liquid formulations described herein may comprise a solubilizing agent component. In some variations the solubilizing agent component is a surfactant. Note that there is some overlap between components that may be solvents and solubilizing agents, and therefore the same component may in some systems be used as either a solvent or a solubilizing agent. A liquid formulation that comprises a therapeutic agent and a component that may be considered either a solvent or a solubilizing agent or surfactant will be considered a solvent if it is playing the role of a solvent; if the component is not playing the role of the solvent, the component may be considered a solubilizing agent or surfactant.

Liquid formulations may optionally further comprise stabilizers, excipients, gelling agents, adjuvants, antioxidants, and/or other components as described herein.

In some variations all components in the liquid formulation, other than the therapeutic agent, are liquid at room temperature.

In some variations, the liquid formulation comprises a release modifying agent. In some variations, the release modifying agent is a film-forming polymer component. The film-forming polymer component may comprise one or more film-forming polymers. Any film-forming polymer may be used in the excipient component. In some variations, the film-forming polymer component comprises a water insoluble film forming polymer. In some variations, the release modifying agent component comprises an acrylic polymer, including but not limited to polymethacrylate, including but not limited to Eudragit RL.

Described herein are compositions and liquid formulations for delivery of the therapeutic agents described in the Therapeutic Agents section. Delivery of therapeutic agents using the compositions and liquid formulations described herein may be used to treat, prevent, inhibit, delay the onset of, or cause the regression of the diseases and conditions described in the Diseases and Conditions section. The compositions and liquid formulations described herein may comprise any of the therapeutic agents described in the Therapeutic Agents section, including but not limited to BOL-303213-X. The compositions and liquid formulations described herein may comprise one or more than one therapeutic agent. Other compositions and liquid formulations in addition to those explicitly described herein may be used.

When the therapeutic agent is BOL-303213-X, the compositions and liquid formulations may be used to maintain an amount of BOL-303213-X in the retina tissue effective to treat wet AMD. In one nonlimiting example, it is believed that a liquid formulation delivering BOL-303213-X to maintain a concentration of BOL-303213-X of about 10 ng/g to about 300 ng/g in the retina choroid tissue over a period of time may be used for the treatment of wet AMD. When the BOL-303213-X is in a liquid formulation that forms a non-dispersed mass, the stated concentration of BOL-303213-X represents the amount that is effectively treating the disease or condition of the eye, and not merely present in the form of the non-dispersed mass. In another nonlimiting example, it is believed that a delivery system delivering BOL-303213-X to maintain a concentration of BOL-303213-X of about 10 ng/g to greater than 300 ng/g in the retina tissues over a period of at least three months time may be used for treatment of wet AMD. Other therapeutically effective amounts of therapeutic agent are also possible, and can be readily determined by one of skill in the art given the teachings herein.

When the therapeutic agent is BOL-303213-X, the compositions and liquid formulations described herein may be used to deliver a dose of BOL-303213-X to a subject, including but not limited to a human subject or to the eye of a subject. In one nonlimiting example, it is believed that a liquid formulation containing a dose of about 10 μg BOL-303213-X in 50 μl HPMC may be used for the treatment of wet AMD. In another nonlimiting example, it is believed that a liquid formulation containing a dose of about 30 μg BOL-303213-X in 50 μl HPMC may be used for treatment of wet AMD.

In some variations the therapeutic agent in the liquid formulation comprises between about 0.01 to about 30% of the total weight of the composition; between about 0.05 to about 15%; between about 0.1 to about 10%; between about 1 to about 5%; or between about 5 to about 15%; between about 8 to about 10%; between about 0.01 to about 1%; between about 0.05 to about 5%; between about 0.1 to about 0.2%; between about 0.2 to about 0.3%; between about 0.3 to about 0.4%; between about 0.4 to about 0.5%; between about 0.5 to about 0.6%; between about 0.6 to about 0.7%; between about 0.7 to about 1%; between about 1 to about 5%; between about 5 to about 10%; between about 15 to about 30%, between about 20 to about 30%; or between about 25 to about 30%.

Those of skill in the art, based on the teachings herein can determine what amount or concentration of a given therapeutic agent is equivalent to an amount or concentration of BOL-303213-X by, for example, administering the therapeutic agent at various amounts or concentrations to a disease model system, such as an in vitro or in vivo model system, and comparing the results in the model system relative to the results of various amounts or concentrations of BOL-303213-X. Those of skill in the art, based on the teachings herein can also determine what amount or concentration of a given therapeutic agent is equivalent to an amount or concentration of BOL-303213-X by reviewing the scientific literature for experiments performed comparing BOL-303213-X to other therapeutic agents. It is understood that even the same therapeutic agent may have a different equivalent level of BOL-303213-X when, for example, a different disease or disorder is being evaluated, or a different type of formulation is used.

For example, in a model for wet AMD, if a therapeutic agent is found to be approximately 10-fold less potent or efficacious than BOL-303213-X in the treatment of wet AMD, a concentration of 10 ng/ml of the therapeutic agent would be equivalent to a 1 ng/ml concentration of BOL-303213-X. Or if a therapeutic agent is found to be approximately 10-fold less potent or efficacious than BOL-303213-X in the treatment of wet AMD, a 10-fold amount of the therapeutic agent would be administered relative to the amount of BOL-303213-X.

The solvent component may comprise, for instance, between about 0.01 to about 99.9% of the total weight of the composition; between about 0.1 to about 99%; between about 25 to about 55%; between about 30 to about 50%; or between about 35 to about 45%; between about 0.1 to about 10%; between about 10 to about 20%; between about 20 to about 30%; between about 30 to about 40%; between about 40 to about 45%; between about 40 to about 45%; between about 45 to about 50%; between about 50 to about 60%; between about 50 to about 70%; between about 70 to about 80%; between about 80 to about 90%; or between about 90 to about 100%.

The solubilizing agent component may comprise, for instance, between about 0.01 to about 30% of the total weight of the composition; between about 0.1 to about 20%; between about 2.5 to about 15%; between about 10 to about 15%; or between about 5 to about 10%; between about 8 to about 12%; between about 10 to about 20%; between about 20 to about 30%.

In some variations the liquid formulations described herein comprise a therapeutic agent and a solvent component. The solvent component may comprise a single solvent or a combination of solvents. The therapeutic agent component may comprise a single therapeutic agent or a combination of therapeutic agents. In some variations, the solvent is glycerin, dimethylsulfoxide, N-methylpyrrolidone, dimethyl acetamide (DMA), dimethyl formamide, glycerol formal, ethoxy diglycol, triethylene glycol dimethyl ether, triacetin, diacetin, corn oil, castor oil, medium chain triglycerides such as Miglyol®, acetyl triethyl citrate (ATC), ethyl lactate, polyglycolated capryl glyceride, γ-butyrolactone, dimethyl isosorbide, benzyl alcohol, benzyl benzoate, ethanol, isopropyl alcohol, polyethylene glycol of various molecular weights, including but not limited to PEG 300 and PEG 400, or propylene glycol, polycaprolactone, propylene carbonate, water or a mixture of one or more thereof.

In some variations the liquid formulations described herein contain no greater than about 250 μl of hydroxypropyl methylcellulose suspension (HPMC). In some variations the liquid formulations described herein contain no greater than about 250 μl, no greater than about 200 μl, no greater than about 150 μl, no greater than about 125 μl, no greater than about 100 μl, no greater than about 75 μl, no greater than about 50 μl, no greater than about 25 μl, no greater than about 20 μL, no greater than about 15 μl, no greater than about 10 μl, no greater than about 7.5 μl, no greater than about 5 μl, no greater than about 2.5 μl, no greater than about 1.0 μl, or no greater than about 0.5 μl of HPMC.

In some variations, the liquid formulations described herein are suspensions, and comprise a therapeutic agent and a diluent component. In some variations, the diluent component comprises one or more components listed herein as solvents or solubilizing agents, wherein the resulting mixture is a suspension.

In some variations the liquid formulation is partly a solution and partly a suspension.

In some variations the liquid formulation is an in situ gelling formulation, and comprises a therapeutic agent and a polymer component, wherein the polymer component may comprise a plurality of polymers. In some variations, the liquid formulation comprises a polymethacrylate polymer. In some variations, the liquid formulation comprises a polyvinylpyrrolidone polymer.

Some variations of liquid formulations include a therapeutic agent or agents such as but not limited to BOL-303213-X between about 0.0001% and about 50% by weight of the total, a solvent between about 50% and about 99.999% by weight of the total, a solubilizing agent including but not limited to a surfactant between about 0.1% and about 25% by weight of the total. In some variations the formulations further comprise stabilizing agents, excipients, adjuvants, or antioxidants, between about 0 and about 40% by weight of the total.

In some variations, a liquid formulation comprises up to about 5% therapeutic agent, including but not limited to BOL-303213-X, per weight of the total; and up to about 99.9% of a solvent component, by weight of the total. In some variations the liquid formulation comprises up to about 5% therapeutic agent, including but not limited to BOL-303213-X, per weight of the total; and up to about 99.9% of a diluent component.

In some variations, a liquid formulation may comprise up to about 5% therapeutic agent, including but not limited to BOL-303213-X, per weight of the total; up to about 10% solvent by weight of the total; and up to about 85% of a solubilizing component, by weight of the total. In some variations the solubilizing component is an aqueous solution of a surfactant.

A plurality of polymers component may comprise, for instance, between about 0.01 to about 30% of the total weight of the composition; between about 0.1 to about 20%; between about 2.5 to about 15%; between about 10 to about 15%; between about 3 to about 5%; between about 5 to about 10%; between about 8 to about 12%; between about 10 to about 20%; or between about 20 to about 30%.

Some variations of liquid formulations includes a therapeutic agent or agents such as but not limited to BOL-303213-X between about 0.01% and about 20% by weight of the total, a solvent component between about 60% and about 98% by weight of the total, and a plurality of polymers, whose combined percentage is between about 0.1% and about 15% by weight of the total. In some variations the formulations further comprise stabilizing agents, excipients, adjuvants, or antioxidants, between about 0 and about 40% by weight of the total.

In some variations, a liquid formulation may comprise about 4% therapeutic agent, including but not limited to BOL-303213-X, per weight of the total; about 91% solvent by weight of the total; and about 5% polymeric component, per weight of the total.

### Liquid Formulations Which Form a Non-Dispersed Mass

One class of liquid formulations described herein forms a non-dispersed mass when placed in an aqueous medium. As used herein, a “non-dispersed mass” refers to the structure formed or shape assumed when the liquid formulation is placed into an environment, relative to the environment in which it is placed. Generally, a non-dispersed mass of a liquid formulation is anything other than a homogeneous distribution of the liquid formulation in the surrounding medium. The non-dispersed mass may, for instance, be indicated by visually inspecting the administered liquid formulation and characterizing its appearance relative to the surrounding medium.

In some variations, the aqueous medium is water. In some variations, the water is deionized, distilled, sterile, or tap water, including but not limited to tap water available at the place of business of Bausch & Lomb Incorporated in Rochester, N.Y.

In some variations, the aqueous medium is an aqueous medium of a subject. In some variations the aqueous medium is an aqueous medium of the eye of a subject, including but not limited to the vitreous of an eye of a subject. In some variations the subject is a human subject. In some variations the subject is a rabbit. In some variations the subject is a monkey.

In some variations the liquid formulation forms a non-dispersed mass when exposed to a certain temperature or range of temperatures, including but not limited to about room temperature, about ambient temperature, about 30° C., about 37° C., or about the temperature of the aqueous medium of the subject.

In some variations the liquid formulation forms a non-dispersed mass when exposed to a certain pH or range of pH, including but not limited to a pH between about 6 and about 8.

In some variations, the non-dispersed mass comprises a gel or gel-like substance.

In some variations the non-dispersed mass comprises an in situ formed solid.

In some variations, the non-dispersed mass comprises a polymer matrix. In some variations, the non-dispersed mass comprises a polymer matrix in which a therapeutic agent is dispersed.

The liquid formulations described herein may generally be of any geometry or shape after administration to a subject or the eye of a subject, including but not limited to a human subject. In some variations, the non-dispersed mass is between about 0.1 and about 5 mm. In some variations, the non-dispersed mass is between about 1 and about 3 mm. The non-dispersed mass-forming liquid formulations may, for instance, appear as a compact spherical mass when administered to the vitreous. In some instances, the liquid formulation may appear as a non-dispersed mass relative to the surrounding medium, wherein the non-dispersed mass is less clearly defined and the geometry is more amorphous than spherical.

The non-dispersed mass-forming liquid formulations described herein may form a non-dispersed mass immediately upon placement in the medium or the non-dispersed mass may form some period of time after placement of the liquid formulation. In some variations the non-dispersed mass forms over the course of about 1, about 2, about 3, about 4, about 5, about 6, or about 7 days. In some variations the non-dispersed mass forms over the course of about 1 week, about 2 weeks, or about 3 weeks.

In some variations, the liquid formulations described herein that form a non-dispersed mass appear as a milky or whitish colored semi-contiguous or semi-solid non-dispersed mass relative to the medium in which it is placed.

In some variations described herein the liquid formulation forms a non-dispersed mass relative to a surrounding medium where the surrounding medium is aqueous. An “aqueous medium” or “aqueous environment” is one that contains at least about 50% water. Examples of aqueous media include but are not limited to water, the vitreous, extracellular fluid, conjunctiva, sclera, the sub-Tenon between the sclera and the conjunctiva, aqueous humor and any tissue or body fluid comprised of at least about 50% of water. Aqueous media include but are not limited to gel structures, including but not limited to those of the conjunctiva and sclera.

In some variations, the liquid formulations described herein form a non-dispersed mass when a test volume of the liquid formulation is placed in the vitreous of a rabbit eye. In some variations the test volume administered to a rabbit eye, and the test volume is equal to the volume of the liquid formulation administered to a subject's, including but not limited to a human subject's eye.

In some variations, the test volume administered to a rabbit eye is equal to the volume administered to the subject's eye multiplied by a scale factor, and the scale factor is equal to the average volume of a rabbit eye divided by the average volume of a subject eye. The “average volume” of an eye, as used herein, refers to the average volume of an eye of a member of similar age of the species under consideration generally, as opposed to the average volume of any particular individual's eye.

In some variations, the test volume administered to the rabbit eye is between about 10 μl and about 50 μl. In some variations, the test volume administered to the rabbit eye is between about 1 μl and about 30 μl. In some variations, the test volume administered to the rabbit eye is between about 50 μl and about 100 μl. In some variations, the test volume administered to the rabbit eye is between about 25 μl and about 50 μl. In some variations, the test volume administered to the rabbit eye is about 50 μl.

In some variations, the liquid formulation that forms a non-dispersed mass when placed in the medium may comprise a therapeutic agent or agents with a concentration of between about 0.01% and about 10% by weight of the total, and a solvent between about 10% and about 99% by weight of the total. In some variations the formulation further comprises a solubilizing agent including but not limited to a surfactant. In some variations the liquid formulation further comprises a stabilizing agent, excipient, adjuvant, or antioxidant, etc., between about 0 and about 40% by weight of the total. In some variations, the therapeutic agent is about 5% by weight of the total, and the solvent component is about 95% by weight of the total.

Whether a liquid formulation exhibits a non-dispersed mass relative to a surrounding medium when present in a subject, including but not limited to a human subject or the eye of a subject may be determined by, for instance, mixing a therapeutic agent with a solvent, administering it to the vitreous of an eye of a subject, including but not limited to a human subject, and comparing the liquid formulation to the surrounding medium.

In some variations, the non-dispersed masses described herein consists of at least about 20%, at least about 30%, at least about 40%, at least about 50%, at least about 60%, at least about 70%, at least about 80%, at least about 90%, or at least about 95% by volume of therapeutic agent when injected into the vitreous of a rabbit eye.

In some variations, upon formation a non-dispersed mass comprising BOL-303213-X, for example, delivers the drug continuously at approximately a constant rate for an extended period of time. Without being bound by theory, it is believed that delivery of BOL-303213-X from a non-dispersed mass in the vitreous depends on dissolution of the BOL-303213-X in the vitreous, which depends in turn on clearance of the drug from the vitreous to other tissues. Without being bound by theory, this release process is believed to maintain a steady-state concentration of BOL-303213-X in the vitreous.

In some variations, it is believed that the liquid formulations will form a visually observable non-dispersed mass when injected into the eye of a subject, including but not limited to a human subject.

In some variations, liquid formulations are believed to form non-dispersed masses when injected subconjunctivally or into the sub-Tenon space. In some variations it is believed that when subconjunctivally administered or administered into the sub-Tenon, the liquid formulation forms a juxtascleral depot adjacent the scleral tissue. That is, it is believed that the therapeutic agent is absorbed into the sclera proximate to the injection site and forms a local concentration of drug in the sclera.

### In Situ Gelling Formulations

An “in situ gelling formulation,” as used herein, refers to a liquid formulation which forms a gel-like non-dispersed mass when the liquid formulation is placed in an aqueous medium, including but not limited to aqueous media that are water, the vitreous of a rabbit eye, and the sub-Tenon between the sclera and the conjunctiva of a rabbit eye. In some variations, an in situ gelling formulation forms a gel-like non-dispersed mass when placed in tap water.

In some variations, the in situ gelling formulation is a suspension prior to placement in an aqueous medium, and forms a gel in situ upon placement in an aqueous medium. In some variations, the in situ gelling formulation is a solution prior to placement in an aqueous medium, and forms a gel in situ upon placement in an aqueous medium. In some variations, the in situ gelling formulation is an emulsion prior to placement in an aqueous medium, and forms a gel in situ upon placement in an aqueous medium. In some variations a gel-like non-dispersed mass forms after placement of the in situ gelling formulation into an aqueous medium, including but not limited to any or all of water, the vitreous, or the sub-Tenon between the sclera and the conjunctiva of an eye. In some variations, the in situ gel is formed of a polymer matrix. In some variations a therapeutic agent is dispersed in the polymer matrix.

In some variations, the in situ gelling formulation comprises one or more polymers. Described herein are various types of polymers, including polymers which are solvents, polymers which are solubilizing agents, polymers which are release modifying agents, polymers which are stabilizing agents, etc. In some variations, any combination of polymers is used wherein the polymers when combined with the therapeutic agent form any or all of a non-dispersed mass, a gel, a hydrogel, or polymeric matrix when placed in an aqueous medium, including but not limited to any or all of water, the vitreous, or the sub-Tenon between the sclera and the conjunctiva.

In some variations, the in situ gelling formulation delivers extended release of therapeutic agents to a subject when administered to the subject.

One liquid formulation described herein comprises a therapeutic agent and a solvent component. The solvent component may comprise a single solvent or a combination of solvents.

In some variations, the solvent is glycerin, dimethylsulfoxide, N-methylpyrrolidone, dimethyl acetamide (DMA), dimethyl formamide, glycerol formal, ethoxy diglycol, triethylene glycol dimethyl ether, triacetin, diacetin, corn oil, castor oil, medium chain triglycerides such as Miglyol®, acetyl triethyl citrate (ATC), ethyl lactate, polyglycolated capryl glyceride, γ-butyrolactone, dimethyl isosorbide, benzyl alcohol, benzyl benzoate, ethanol, isopropyl alcohol, polyethylene glycol of various molecular weights, including but not limited to PEG 300 and PEG 400, or propylene glycol, polycaprolactone, propylene carbonate, water or a mixture of one or more thereof.

In some variations, the solvent is polyethylene glycol. Polyethylene glycol is known by various names and is available in various preparations, including but not limited to macrogels, macrogel 400, macrogel 1500, macrogel 4000, macrogel 6000, macrogel 20000, macrogola, breox PEG; carbowax; carbowax sentry; Hodag PEG; Lipo; Lipoxol; Lutrol E; PEG; Pluriol E; polyoxyethylene glycol, and α-Hydro-ω-hydroxy-poly(oxy-1,2-ethanediyl).

### Compositions and Liquid Formulations for Delivery of Therapeutic Agents

The compositions and liquid formulations described herein may be used to deliver amounts of the therapeutic agents effective for treating, preventing, inhibiting, delaying onset of, or causing the regression of the diseases and conditions described in the Diseases and Conditions section. In some variations the compositions and liquid formulations described herein deliver one or more therapeutic agents over an extended period of time.

An “effective amount,” which is also referred to herein as a “therapeutically effective amount,” of a therapeutic agent for administration as described herein is that amount of the therapeutic agent that provides the therapeutic effect sought when administered to the subject, including but not limited to a human subject. The achieving of different therapeutic effects may require different effective amounts of therapeutic agent. For example, the therapeutically effective amount of a therapeutic agent used for preventing a disease or condition may be different from the therapeutically effective amount used for treating, inhibiting, delaying the onset of, or causing the regression of the disease or condition. In addition, the therapeutically effective amount may depend on the age, weight, and other health conditions of the subject as is well known to those versed in the disease or condition being addressed. Thus, the therapeutically effective amount may not be the same in every subject to which the therapeutic agent is administered.

An effective amount of a therapeutic agent for treating, preventing, inhibiting, delaying the onset of, or causing the regression of a specific disease or condition is also referred to herein as the amount of therapeutic agent effective to treat, prevent, inhibit, delay the onset of, or cause the regression of the disease or condition.

To determine whether a level of therapeutic agent is a “therapeutically effective amount” to treat, prevent, inhibit, delay on set of, or cause the regression of the diseases and conditions described in the Diseases and conditions section, liquid formulations may be administered in animal models for the diseases or conditions of interest, and the effects may be observed. In addition, dose ranging human clinical trials may be conducted to determine the therapeutically effective amount of a therapeutic agent.

Generally, the therapeutic agent may be formulated in any composition or liquid formulation capable of delivery of a therapeutically effective amount of the therapeutic agent to a subject or to the eye of a subject for the required delivery period. Compositions include liquid formulations.

### Solubilization of Therapeutic Agents

One composition or liquid formulation that may be used is a composition or liquid formulation in which the therapeutic agent is dissolved in a solvent component. Generally, any solvent which has the desired effect may be used in which the therapeutic agent dissolves. In some variations the solvent is aqueous. In some variations the solvent is non-aqueous. An “aqueous solvent” is a solvent that contains at least about 50% water.

Generally, any concentration of solubilized therapeutic agent that has the desired effect can be used. The solvent component may be a single solvent or may be a mixture of solvents. Solvents and types of solutions are well known to those versed in such drug delivery technologies. See for example, Remington: The Science and Practice of Pharmacy, Twentieth Edition, Lippincott Williams & Wilkins; 20th edition (Dec. 15, 2000); Ansel's Pharmaceutical Dosage Forms and Drug Delivery Systems, Eighth Edition, Lippincott Williams & Wilkins (August 2004); Handbook Of Pharmaceutical Excipients 2003, American Pharmaceutical Association, Washington, D.C., USA and Pharmaceutical Press, London, UK; and Strickley, Solubilizing Excipients in Oral and Injectable Formulations, Pharmaceutical Research, Vol. 21, No. 2, February 2004.

As noted previously, some solvents may also serve as solubilizing agents.

Solvents that may be used include but are not limited to glycerin, dimethylsulfoxide, N-methylpyrrolidone, dimethyl acetamide (DMA), dimethyl formamide, glycerol formal, ethoxy diglycol, triethylene glycol dimethyl ether, triacetin, diacetin, corn oil, castor oil, medium chain triglycerides such as Miglyol®, acetyl triethyl citrate (ATC), ethyl lactate, polyglycolated capryl glyceride, γ-butyrolactone, dimethyl isosorbide, benzyl alcohol, benzyl benzoate, ethanol, isopropyl alcohol, polyethylene glycol of various molecular weights, including but not limited to PEG 300 and PEG 400, or propylene glycol, polycaprolactone, propylene carbonate, water or a mixture of one or more thereof, combinations of any one or more of the foregoing, or analogs or derivatives of any one or more of the foregoing.

In some variations, the solvent is a polyethylene glycol. Polyethylene glycol is known by various names and is available in various preparations, including but not limited to macrogels, macrogel 400, macrogel 1500, macrogel 4000, macrogel 6000, macrogel 20000, macrogola, breox PEG; carbowax; carbowax sentry; Hodag PEG; Lipo; Lipoxol; Lutrol E; PEG; Pluriol E; polyoxyethylene glycol, and α-Hydro-ω-hydroxy-poly(oxy-1,2-ethanediyl).

In some variations the polyethylene glycol is a liquid PEG, and is one or more of PEG 300 or PEG 400.

Other solvents include an amount of a C₆-C₂₄ fatty acid sufficient to solubilize a therapeutic agent.

Phospholipid solvents may also be used, such as lecithin, phosphatidylcholine, or a mixture of various diglycerides of stearic, palmitic, and oleic acids, linked to the choline ester of phosphoric acid; hydrogenated soy phosphatidylcholine (HSPC), distearoylphosphatidylglycerol (DSPG), L-α-dimyristoylphosphatidylcholine (DMPC), L-α-dimyristoylphosphatidylglycerol (DMPG).

Further examples of solvents include, for example, components such as alcohols, propylene glycol, polyethylene glycol of various molecular weights, propylene glycol esters, propylene glycol esterified with fatty acids such as oleic, stearic, palmic, capric, linoleic, etc; medium chain mono-, di-, or triglycerides, long chain fatty acids, naturally occurring oils, and a mixture thereof. The oily components for the solvent system include commercially available oils as well as naturally occurring oils. The oils may further be vegetable oils or mineral oils. The oils can be characterized as non-surface active oils, which typically have no hydrophilic lipophilic balance value. Commercially available substances comprising medium chain triglycerides include, but are not limited to, Captex 100, Captex 300, Captex 355, Miglyol 810, Miglyol 812, Miglyol 818, Miglyol 829, and Dynacerin 660. Propylene glycol ester compositions that are commercially available encompass Captex 200 and Miglyol 840, and the like. The commercial product, Capmul MCM, comprises one of many possible medium chain mixtures comprising monoglycerides and diglycerides.

Other solvents include naturally occurring oils such as peppermint oil, and seed oils. Exemplary natural oils include oleic acid, castor oil, safflower seed oil, soybean oil, olive oil, sunflower seed oil, sesame oil, and peanut oil. Soy fatty acids may also be used. Examples of fully saturated non-aqueous solvents include, but are not limited to, esters of medium to long chain fatty acids (such as fatty acid triglycerides with a chain length of about C₆ to about C₂₄). Hydrogenated soybean oil and other vegetable oils may also be used. Mixtures of fatty acids may be split from the natural oil (for example coconut oil, palm kernel oil, babassu oil, or the like) and refined. In some embodiments, medium chain (about C₈ to about C₁₂) triglycerides, such as caprylic/capric triglycerides derived from coconut oil or palm seed oil, may be used. Medium chain mono- and diglycerides may also be used. Other fully saturated non-aqueous solvents include, but are not limited to, saturated coconut oil (which typically includes a mixture of lauric, myristic, palmitic, capric and caproic acids), including those sold under the Miglyol™ from Huls and bearing trade designations 810, 812, 829 and 840. Also noted are the NeoBee™ products sold by Stepan. Non-aqueous solvents include isopropyl myristate. Examples of synthetic oils include triglycerides and propylene glycol diesters of saturated or unsaturated fatty acids having 6 to 24 carbon atoms such as, for example hexanoic acid, octanoic (caprylic), nonanoic (pelargonic), decanoic (capric), undecanoic, lauric, tridecanoic, tetradecanoic (myristic), pentadecanoic, hexadecanoic (palmitic), heptadecanoic, octadecanoic (stearic), nonadecanoic, heptadecanoic, eicosanoic, heneicosanoic, docosanoic and lignoceric acids, and the like. Examples of unsaturated carboxylic acids include oleic, linoleic and linolenic acids, and the like. The non-aqueous solvent can comprise the mono-, di- and triglyceryl esters of fatty acids or mixed glycerides and/or propylene glycol mono- or diesters wherein at least one molecule of glycerol has been esterified with fatty acids of varying carbon atom length. A non-limiting example of a “non-oil” useful as a solvent is polyethylene glycol.

Exemplary vegetable oils include cottonseed oil, corn oil, castor oil, sesame oil, soybean oil, olive oil, fractionated coconut oil, peanut oil, sunflower oil, safflower oil, almond oil, avocado oil, palm oil, palm kernel oil, babassu oil, beechnut oil, linseed oil, rape oil and the like. Mono-, di-, and triglycerides of vegetable oils, including but not limited to corn, may also be used.

Polyvinyl pyrrolidone (PVP), cross-linked or not, may also be used as a solvent. Further solvents include but are not limited to C₆-C₂₄ fatty acids, oleic acid, Imwitor 742, Capmul, F68, F68 (Lutrol), PLURONIICS including but not limited to PLURONICS F108, F127, and F68, Poloxamers, Jeffamines), Tetronics, F127; cyclodextrins such as α-cyclodextrin, β-cyclodextrin, hydroxypropyl-β-cyclodextrin, sulfobutylether-β-cyclodextrin (Captisol); CMC, polysorbitan 20, Cavitron, polyethylene glycol of various molecular weights including but not limited to PEG 300 and PEG 400.

Beeswax and d-α-tocopherol (Vitamin E) may also be used as solvents.

Solvents for use in the liquid formulations can be determined by a variety of methods known in the art, including but not limited to (1) theoretically estimating their solubility parameter values and choosing the ones that match with the therapeutic agent, using standard equations in the field; and (2) experimentally determining the saturation solubility of therapeutic agent in the solvents, and choosing the ones that exhibit the desired solubility.

### Solubilizing Agents

Generally, any solubilizing agent or combination of solubilizing agents may be used in the liquid formulations described herein.

In some variations, the solubilizing agent is a surfactant or combination of surfactants. Many surfactants are possible. Combinations of surfactants, including combinations of various types of surfactants, may also be used. For instance, surfactants which are nonionic, anionic (i.e. soaps, sulfonates), cationic (i.e. CTAB), zwitterionic, polymeric or amphoteric may be used.

Surfactants that can be used may be determined by mixing a therapeutic agent of interest with a putative solvent and a putative surfactant, and observing the characteristics of the formulation after exposure to a medium.

Examples of surfactants include but are not limited to fatty acid esters or amides or ether analogues, or hydrophilic derivatives thereof, monoesters or diesters, or hydrophilic derivatives thereof, or mixtures thereof, monoglycerides or diglycerides, or hydrophilic derivatives thereof, or mixtures thereof, mixtures having enriched mono- and/or diglycerides, or hydrophilic derivatives thereof, surfactants partially derivatized with a hydrophilic moiety; monoesters or diesters or multiple-esters of other alcohols, polyols, saccharides or oligosaccharides or polysaccharides, oxyalkylene oligomers or polymers or block polymers, or hydrophilic derivatives thereof, or the amide analogues thereof, fatty acid derivatives of amines, polyamines, polyimines, aminoalcohols, aminosugars, hydroxyalkylamines, hydroxypolyimines, peptides, polypeptides, or the ether analogues thereof.

Hydrophilic Lipophilic Balance (“HLB”) is an expression of the relative simultaneous attraction of a surfactant for water and oil (or for the two phases of the emulsion system being considered).

Surfactants are characterized according to the balance between the hydrophilic and lipophilic portions of their molecules. The hydrophilic-lipophilic balance (HLB) number indicates the polarity of the molecule in an arbitrary range of 1-40, with the most commonly used emulsifiers having a value between 1-20. The HLB increases with increasing hydrophilicity.

Surfactants that may be used include but are not limited to those with an HLB greater than 10, 11, 12, 13 or 14. Examples of surfactants include polyoxyethylene products of hydrogenated vegetable oils, polyethoxylated castor oils or polyethoxylated hydrogenated castor oil, polyoxyethylene-sorbitan-fatty acid esters, polyoxyethylene castor oil derivatives and the like, for example, Nikkol HCO-50, Nikkol HCO-35, Nikkol HCO-40, Nikkol HCO-60 (from Nikko Chemicals Co. Ltd.); Cremophor (from BASF) such as Cremophor RH40, Cremophor RH60, Cremophor EL, TWEENs (from ICI Chemicals) e.g., TWEEN 20, TWEEN 21, TWEEN 40, TWIEEN 60, TWEEN 80, TWEEN 81, Cremophor RH 410, Cremophor RH 455 and the like.

The surfactant component may be selected from compounds having at least one ether formed from at least about 1 to 100 ethylene oxide units and at least one fatty alcohol chain having from at least about 12 to 22 carbon atoms; compounds having at least one ester formed from at least about 1 to 100 ethylene oxide units and at least one fatty acid chain having from at least about 12 to 22 carbon atoms; compounds having at least one ether, ester or amide formed from at least about 1 to 100 ethylene oxide units—and at least one vitamin or vitamin derivative; and combinations thereof

Other examples of surfactants include Lumulse GRH-40, TGPS, Polysorbate-80 (TWEEN-80), Polysorbate-20 (TWEEN-20), polyoxyethylene (20) sorbitan mono-oleate), glyceryl glycol esters, polyethylene glycol esters, polyglycolyzed glycerides, and the like, or mixtures thereof, polyethylene sorbitan fatty acid esters, polyoxyethylene glycerol esters, such as Tagat TO, Tagat L, Tagat I, Tagat 12 and Tagat 0 (commercially available from Goldschmidt Chemical Co., Essen, Germany); ethylene glycol esters, such as glycol stearate and distearate; propylene glycol esters, such as propylene glycol myristate; glyceryl esters of fatty acids, such as glyceryl stearates and monostearates; sorbitan esters, such as spans and TWEENs; polyglyceryl esters, such as polyglyceryl 4-oleate; fatty alcohol ethoxylates, such as Brij type emulsifiers; ethoxylated propoxylated block copolymers, such as poloxamers; polyethylene glycol esters of fatty acids, such as PEG 300 linoleic glycerides or Labrafil 2125 CS, PEG 300 oleic glycerides or Labrafil M 1944 CS, PEG 400 caprylic/capric glycerides or Labrasol, and PEG 300 caprylic/capric glycerides or Softigen 767; cremophors, such as Cremophor B, polyoxyl 35 castor oil or Cremophor EL, Cremophor EL-P, Cremophor RH 40P, polyoxyl 40 hydrogenated castor oil, Cremophor RH40; polyoxyl 60 hydrogenated castor oil or Cremophor RH 60, glycerol mono caprylate/caprate, such as Capmul CM 10; polyoxyethylated fatty acids (PEG-stearates, PED-laurates, Brij®), polyoxylated glycerides of fatty acid, polyoxylated glycerol fatty acid esters, i.e. Solutol HS-15; PEG-ethers (Miij®), sorbitan derivatives (TWEENs), sorbitan monooleate or Span 20, aromatic compounds (Tritons®), PEG-glycerides (PECEOL™), PEG-PPG (polypropylene glycol) copolymers (PLURONICS including but not limited to PLURONICS F108, F 127, and F68, Poloxamers, Jeffamines), Tetronics, Polyglycerines, PEG-tocopherols, PEG-LICOL 6-oleate; propylene glycol derivatives, sugar and polysaccharide alkyl and acyl derivatives (octylsucrose, sucrose stearate, lauroyldextran etc.) and/or a mixture thereof, surfactants based on an oleate or laureate ester of a polyalcohol copolymerized with ethylene oxide; Labrasol Gelucire 44/14; polyoxyethylene stearates; saturated polyglycolyzed glycerides; or poloxamers; all of which are commercially available. Polyoxyethylene sorbitan fatty acid esters can include polysorbates, for example, polysorbate 20, polysorbate 40, polysorbate 60, and polysorbate 80. Polyoxyethylene stearates can include polyoxyl 6 stearate, polyoxyl 8 stearate, polyoxyl 12 stearate and polyoxyl 20 stearate. Saturated polyglycolyzed glycerides are, for example, GELUCIRE 44/14 or GELUCIRE TM 50/13 (Gattefosse, Westwood, N.J., U.S.A.). Poloxamers used herein include poloxamer 124 and poloxamer 188.

Surfactants include d-α-tocopheryl polyethylene glycol 1000 succinate (TPGS), polyoxyl 8 stearate (PEG 400 monostearate), polyoxyl 40 stearate (PEG 1750 monostearate) and peppermint oil.

In some variations, surfactants having an HLB lower than 10 are used. Such surfactants may optionally be used in combination with other surfactants as co-surfactants. Examples of some surfactants, mixtures, and other equivalent compositions having an HLB less than or equal to 10 are propylene glycols, glyceryl fatty acids, glyceryl fatty acid esters, polyethylene glycol esters, glyceryl glycol esters, polyglycolyzed glycerides and polyoxyethyl stearyl ethers. Propylene glycol esters or partial esters form the composition of commercial products, such as Lauroglycol FCC, which contains propylene glycol laureate. The commercially available excipient Maisine 35-1 comprises long chain fatty acids, for example glyceryl linoleate. Products, such as Acconon E, which comprise polyoxyethylene stearyl ethers, may also be used. Labrafil M 1944 CS is one example of a surfactant wherein the composition contains a mixture of glyceryl glycol esters and polyethylene glycol esters.

### Solubilizing Agents for BOL-303213-X

Many solubilizing agents may be used for BOL-303213-X, including but not limited to those in the solubilizing agents section above.

In some variations the solubilizing agent is a surfactant. In some variations, the surfactant may be a polymeric surfactant including but not limited to PLURONICS F108, F127, and F68, and Tetronics. As noted herein, some solvents may also serve as surfactants. Those of ordinary skill in the art will find it routine to identify which solubilizing agents and surfactants may be used for BOL-303213-X given the teachings herein.

### Viscosity Modifying Agents

The liquid formulations described herein may be administered with or further comprise a viscosity modifying agent.

One exemplary viscosity modifying agent that may be used is hyaluronic acid. Hyaluronic acid is a glycosaminoglycan. It is made of a repetitive sequence of glucuronic acid and glucosamine. Hyaluronic acid is present in many tissues and organs of the body, and contributes to the viscosity and consistency of such tissues and organs. Hyaluronic acid is present in the eye, including the vitreous of the eye, and along with collagen contributes to the viscosity thereof. The liquid formulations described herein may further comprise or be administered with hyaluronic acid.

Other non-limiting examples of viscosity modifying agents include polyalkylene oxides, glycerol, carboxymethyl cellulose, sodium alginate, chitosan, dextran, dextran sulfate and collagen. These viscosity modifying agents can be chemically modified.

Other viscosity modifying agents that may be used include but are not limited to carrageenan, cellulose gel, colloidal silicon dioxide, gelatin, propylene carbonate, carbonic acid, alginic acid, agar, carboxyvinyl polymers or carbomers and polyacrylamides, acacia, ester gum, guar gum, gum arabic, ghatti, gum karaya, tragacanth, terra, pectin, tamarind seed, larch arabinogalactan, alginates, locust bean, xanthan gum, starch, veegum, tragacanth, polyvinyl alcohol, gellan gum, hydrocolloid blends, and povidone. Other viscosity modifying agents known in the art can also be used, including but not limited to sodium carboxymethyl cellulose, algin, carageenans, galactomarmans, hydroxyethyl cellulose, hydroxyethyl methyl cellulose, hydroxypropyl methylcellulose, hydroxypropyl cellulose, polyethylene glycol, polyvinylpyrrolidone, sodium carboxymethyl chitin, sodium carboxymethyl dextran, sodium carboxymethyl starch, xanthan gum, and zein.

The therapeutic agents for use as described herein, such as BOL-303213-X, may be subjected to conventional pharmaceutical operations, such as sterilization and compositions containing the therapeutic agent may also contain conventional adjuvants, such as preservatives, stabilizers, wetting agents, emulsifiers, buffers etc. The therapeutic agents may also be formulated with pharmaceutically acceptable excipients for clinical use to produce a pharmaceutical composition. Formulations for ocular administration may be presented as a solution, suspension, particles of solid material, a discrete mass of solid material, incorporated within a polymer matrix, liquid formulations or in any other form for ocular administration. The therapeutic agents may be used to prepare a medicament to treat, prevent, inhibit, delay onset, or cause regression of any of the conditions described herein. In some variations, the therapeutic agents may be used to prepare a medicament to treat any of the conditions described herein.

A composition containing a therapeutic agent such as BOL-303213-X may contain one or more adjuvants appropriate for the indicated route of administration. Adjuvants with which the therapeutic agent may be admixed with include but are not limited to lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinylpyrrolidone, and/or polyvinyl alcohol. When a solubilized formulation is required the therapeutic agent may be in a solvent including but not limited to polyethylene glycol of various molecular weights, propylene glycol, carboxymethyl cellulose colloidal solutions, methanol, ethanol, DMSO, corn oil, peanut oil, cottonseed oil, sesame oil, castor oil, tragacanth gum, and/or various buffers. Other adjuvants and modes of administration are well known in the pharmaceutical art and may be used in the practice of the methods, compositions and liquid formulations described herein. The carrier or diluent may include time delay material, such as glyceryl monostearate or glyceryl distearate alone or with a wax, or other materials well known in the art. The formulations for use as described herein may also include gel formulations, erodible and non-erodible polymers, micro spheres, and liposomes.

Other adjuvants and excipients that may be used include but are not limited to C₈-C₁₀ fatty acid esters such as softigen 767, polysorbate 80, PLURONICS, Tetronics, Miglyol, and Transcutol.

Additives and diluents normally utilized in the pharmaceutical arts can optionally be added to the pharmaceutical composition and the liquid formulation. These include thickening, granulating, dispersing, and stabilizing agents, including pH stabilizers, other excipients, anti-oxidants (e.g., tocopherol, BHA, BHT, TBHQ, tocopherol acetate, ascorbyl palmitate, ascorbic acid propyl gallate, and the like), preservatives (e.g., parabens), and the like. Exemplary preservatives include, but are not limited to, benzylalcohol, ethylalcohol, benzalkonium chloride, phenol, chlorobutanol, stabilized chlorine dioxide and the like. Some useful antioxidants provide oxygen or peroxide inhibiting agents for the formulation and include, but are not limited to, butylated hydroxytoluene, butylhydroxyanisole, propyl gallate, ascorbic acid palmitate, α-tocopherol, and the like. Thickening agents, such as lecithin, hydroxypropylcellulose, aluminum stearate, and the like, may improve the texture of the formulation.

In addition, a viscous polymer may be added to the suspension, assisting the localization and ease of placement and handling. In some uses of the liquid formulation, a pocket in the sclera may be surgically formed to receive an injection of the liquid formulations. The hydrogel structure of the sclera can act as a rate-controlling membrane.

Particles of therapeutic agent substance for forming a suspension can be produced by known methods including but not limited to via ball milling, for example by using ceramic beads. For example, a Cole Parmer ball mill such as Labmill 8000 may be used with 0.8 mm YTZ ceramic beads available from Tosoh or Norstone Inc.

The formulations may conveniently be presented in unit dosage form and may be prepared by conventional pharmaceutical techniques. Such techniques include the step of bringing into association the therapeutic agent and the pharmaceutical carrier(s) or excipient(s). The formulations may be prepared by uniformly and intimately bringing into association the active ingredient with liquid carriers or finely divided solid carriers or both, and then, if necessary, shaping the product.

In some variations, the formulations described herein are provided in one or more unit dose forms, wherein the unit dose form contains an amount of a liquid formulation described herein that is effective to treat or prevent the disease or condition for which it is being administered. In some variations, the formulations described herein are provided in one or more unit dose forms, wherein the unit dose form contains an amount of a liquid BOL-303213-X formulation described herein that is effective to treat or prevent the disease or condition for which it is being administered.

In some embodiments, the unit dose form is prepared in the concentration at which it will be administered. In some variations, the unit dose form is diluted prior to administration to a subject. In some variations, a liquid formulation described herein is diluted in an aqueous medium prior to administration to a subject. In some variations the aqueous medium is an isotonic medium. In some variations, a liquid formulation described herein is diluted in an non-aqueous medium prior to administration to a subject.

In a further aspect, provided herein are kits comprising one or more unit dose forms as described herein. In some embodiments, the kit comprises one or more of packaging and instructions for use to treat one or more diseases or conditions. In some embodiments, the kit comprises a diluent which is not in physical contact with the formulation or pharmaceutical formulation. In some embodiments, the kit comprises any of one or more unit dose forms described herein in one or more sealed vessels. In some embodiments, the kit comprises any of one or more sterile unit dose forms.

### Routes of Administration

The compositions, methods, and liquid formulations described herein deliver one or more therapeutic agents to a subject, including but not limited to a human subject.

In some variations, the compositions, methods, and liquid formulations described herein deliver one or more therapeutic agents to an aqueous medium of a human subject.

In some variations, the compositions, methods, and liquid formulations described herein deliver one or more therapeutic agents to an aqueous medium in or proximal to an area where a disease or condition is to be treated, prevented, inhibited, onset delayed, or regression caused.

In some variations, the compositions, methods, and liquid formulations described herein deliver one or more therapeutic agents to an eye of a subject, including the macula and the retina tissues, in an amount and for a duration effective to treat, prevent, inhibit, delay the onset of, or cause the regression of the diseases and conditions described in the Diseases and Conditions section.

As a non-limiting example, the compositions, liquid formulations, and methods described herein may be administered to the vitreous, aqueous humor, sclera, conjunctiva, the sub-Tenon between the sclera and conjunctiva, the retina, macula, or other area in or proximate to the eye of a subject, either by direct administration to these tissues or by periocular routes, in amounts and for a duration effective to treat, prevent, inhibit, delay the onset of, or cause the regression of CNV and wet AMD. The effective amounts and durations may be different for each of treating, preventing, inhibiting, delaying the onset of, or causing the regression of CNV and wet AMD, and for each of the different sites of delivery.

Intravitreal administration is more invasive than some other types of ocular procedures. Because of the potential risks of adverse effects, intravitreal administration may not be optimal for treatment of relatively healthy eyes. By contrast, periocular administration, such as sub-Tenon or subconjunctival administration, is much less invasive than intravitreal administration. When a therapeutic agent is delivered by a periocular route, it may be possible to treat patients with healthier eyes than could be treated using intravitreal administration. In some variations, sub-Tenon or subconjunctival injection is used to prevent or delay onset of a disease or condition of the eye, where the eye of the subject has visual acuity of 20/40 or better.

“Subconjunctival” placement or injection, as used herein, refers to placement or injection between the conjunctiva and Tenon's capsule. Subconjunctival is sometimes referred to herein as “sub-conj” administration. “Sub-Tenon” placement of injection, as used herein, refers to placement or injection in the region between Tenon's capsule and the sclera. Routes of administration that may be used to administer a liquid formulation include but are not limited to placement of the liquid formulation, for example by injection, into an aqueous medium in the subject, including but not limited to placement, including but not limited to by injection, into the eye of a subject, including but not limited to a human subject.

Compositions and liquid formulations comprising therapeutic agent can be administered directly to the eye using a variety of procedures, including but not limited to procedures in which (1) the therapeutic agent is administered by injection using a syringe and hypodermic needle, (2) a specially designed device is used to inject the therapeutic agent, (3) prior to injection of the therapeutic agent, a pocket is surgically formed within the sclera to serve as a receptacle for the therapeutic agent or therapeutic agent composition, (4) the therapeutic agent is administered in an intravitreal device, (5) the therapeutic agent is administered in a scleral plug or tack. For example, in one administration procedure a surgeon forms a pocket within the sclera of the eye followed by injection of a solution or liquid formulation comprising the therapeutic agent into the pocket.

Other administration procedures include, but are not limited to procedures in which (1) a formulation of the therapeutic agent is injected through a specially designed curved cannula to place the therapeutic agent directly against a portion of the eye, (2) a compressed form of the therapeutic agent is placed directly against a portion of the eye, (3) the therapeutic agent is inserted into the sclera by a specially designed injector or inserter, (4) the liquid formulation comprising the therapeutic agent is incorporated within a polymer, (5) a surgeon makes a small conjunctival incision through which to pass a suture and any therapeutic agent delivery structure so as to secure the structure adjacent to the sclera, (6) a needle is used for injection directly into the vitreous of an eye, or into any other site described.

The liquid formulations described herein may be used directly, for example, by injection, as an elixir, for topical administration including but not limited to via eye drops.

### Delivery by Injection

One method that may be used to deliver the compositions and liquid formulations described herein is delivery by injection. In this method compositions and liquid formulations may be injected into a subject, including but not limited to a human subject, or into a position in or proximate to an eye of the subject for delivery to a subject or to the eye of a subject. Injection includes but is not limited to intraocular and periocular injection. Nonlimiting examples of positions that are in or proximate to an eye of a subject are as follows.

Injection of therapeutic agent into the vitreous may provide a high local concentration of therapeutic agent in the vitreous and retina. Further, it has been found that in the vitreous the clearance half-lives of drugs increases with molecular weight.

Intracameral injection, or injection into the anterior chamber of they eye, may also be used. In one example, up to about 100 μl may be injected intracamerally.

Periocular routes of delivery may deliver therapeutic agent to the retina without some of the risks of intravitreal delivery. Periocular routes include but are not limited to subconjunctival, sub-Tenon, retrobulbar, peribulbar and posterior juxtascleral delivery. A “periocular” route of administration means placement near or around the eye.

In some variations the liquid formulations described herein are administered intraocularly. Intraocular administration includes placement or injection within the eye, including in the vitreous.

Subconjunctival injection may be by injection of therapeutic agent underneath the conjunctiva, or between the sclera and conjunctiva. In one example, up to about 100 μl may be injected subconjunctivally. As one nonlimiting example, a needle of up to about 22 to about 30 gauge and about 30 mm long may be used.

Sub-Tenon injection may be by injection of therapeutic agent between the Tenon's capsule and the sclera. In one example, up to about 10 ml may be injected into the sub-Tenon. As one nonlimiting example, a blunt-tipped cannula about 2.5 cm long may be used.

Retrobulbar injection refers to injection into the conical compartment of the four rectus muscles and their intermuscular septa, behind the globe of the eye. In one example, up to about 5 ml may be injected retrobulbarly. As one nonlimiting example, a blunt needle of about 25- or about 27-gauge may be used.

Peribulbar injection may be at a location external to the confines of the four rectus muscles and their intramuscular septa, i.e., outside of the muscle cone. A volume of, for example, up to about 10 ml may be injected peribulbarly. As one nonlimiting example, a blunt-tipped cannula about 1.25 inches long and about 25-gauge may be used.

Posterior juxtascleral delivery refers to placement of a therapeutic agent near and above the macula, in direct contact with the outer surface of the sclera, and without puncturing the eyeball. As one non-limiting example, a blunt-tipped curved cannula, specially designed at 56°, is used to place the therapeutic agent in an incision in the sclera.

In some variations the liquid formulations described herein are injected intraocularly. Intraocular injection includes injection within the eye.

Sites to which the compositions and liquid formulations may be administered include but are not limited to the vitreous, aqueous humor, sclera, conjunctiva, sub-Tenon between the sclera and conjunctiva, the retina tissues, macula, or other area in or proximate to the eye of a subject. Methods that may be used for placement of the compositions and liquid formulations include but are not limited to injection.

In one method that may be used, the therapeutic agent is dissolved in a solvent or solvent mixture and then injected into or proximate to the vitreous, aqueous humor, sclera, conjunctiva, sub-Tenon between the sclera and conjunctiva, the retina, macula, or other area in or proximate to the eye of a subject, or other medium of a subject, according to any of the procedures mentioned above. In one such method that may be used, the therapeutic agent is BOL-303213-X in a liquid formulation.

When the therapeutic agent is BOL-303213-X, the compositions and liquid formulations may be used to deliver or maintain an amount of BOL-303213-X in tissues of the eye, including without limitation retina, choroid, or the vitreous, which amount is effective to treat AMD. In one nonlimiting example, it is believed that a liquid formulation delivering BOL-303213-X in an amount capable of providing a concentration of BOL-303213-X of about 0.1 pg/ml to about 2 μg/ml in the vitreous may be used for treatment of wet AMD. In some nonlimiting examples, it is believed that a liquid formulation delivering a concentration of BOL-303213-X of about 0.1 pg/mg to about 1 μg/mg in the retina tissues may be used for treatment of wet AMD. Other effective concentrations are readily ascertainable by those of skill in the art based on the teachings described herein.

### Method of Preparing Liquid Formulations

One nonlimiting method that may be used for preparing the liquid formulations described herein, including but not limited to liquid formulations comprising BOL-303213-X, is by mixing a solvent and a therapeutic agent together at room temperature or at slightly elevated temperature until a solution or suspension is obtained, with optional use of a sonicator, and then cooling the formulation. Other components including but not limited to those described above may then be mixed with the formulation. Other preparation methods that may be used are described herein including in the examples, and those of skill in the art will be able to select other preparation methods based on the teachings herein.

### Extended Delivery of Therapeutic Agents

Described herein are compositions and liquid formulations showing in vivo delivery or clearance profiles with one or more of the following characteristics. The delivery or clearance profiles are for clearance of the therapeutic agent in vivo after injection of the composition or liquid formulations subconjunctivally or into the vitreous of a rabbit eye. In some variations, the delivery or clearance profiles are for clearance of BOL-303213-X in vivo after injection of the composition or liquid formulations subconjunctivally or into the vitreous of a rabbit eye. The volume of the rabbit vitreous is approximately 25-40% of the volume of the human vitreous.

For treatment, prevention, inhibition, delaying the onset of, or causing the regression of certain diseases or conditions, it may be desirable to maintain delivery of a therapeutically effective amount of the therapeutic agent for an extended period of time. Depending on the disease or condition being treated, prevented, inhibited, having onset delayed, or being caused to regress this extended period of time may be at least about 1 week, at least about 2 weeks, at least about 3 weeks, at least about 1 month, at least about 3 months, at least about 6 months, at least about 9 months, at least about 12 months, at least about 18 months, or at least about 24 months. Generally, however, any extended period of delivery may be possible. A therapeutically effective amount of agent may be delivered for an extended period by a liquid formulation or composition that maintains for the extended period a concentration of agent in a subject or an eye of a subject sufficient to deliver a therapeutically effective amount of agent for the extended time.

Delivery of a therapeutically effective amount of the therapeutic agent for an extended period may be achieved via placement of one composition or liquid formulation or may be achieved by application of two or more doses of composition or liquid formulations. As a non-limiting example of such multiple applications, maintenance of the therapeutic amount of BOL-303213-X for 3 months for treatment, prevention, inhibition, delay of onset, or cause of regression of wet AMD may be achieved by application of one liquid formulation or composition delivering a therapeutic amount for 3 months or by sequential application of a plurality of liquid formulations or compositions. The optimal dosage regime will depend on the therapeutic amount of the therapeutic agent needing to be delivered, and the period over which it need be delivered. Those versed in such extended therapeutic agent delivery dosing will understand how to identify dosing regimes that may be used based on the teachings provided herein.

When using certain therapeutic agents or for the treatment, prevention, inhibition, delaying the onset of, or causing the regression of certain diseases, it may be desirable for delivery of the therapeutic agent not to commence immediately upon placement of the liquid formulation or composition into the eye region, but for delivery to commence after some delay. For example, but in no way limiting, such delayed release may be useful where the therapeutic agent inhibits or delays wound healing and delayed release is desirable to allow healing of any wounds occurring upon placement of the liquid formulation or composition. Depending on the therapeutic agent being delivered and/or the diseases and conditions being treated, prevented, inhibited, onset delayed, and regression caused this period of delay before delivery of the therapeutic agent commences may be about 1 hour, about 6 hours, about 12 hours, about 18 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, or about 42 days. Other delay periods may be possible. Delayed release formulations that may be used are known to people versed in the technology.

As described herein, the dosage of the therapeutic agent will depend on the condition being addressed, whether the condition is to be treated, prevented, inhibited, have onset delayed, or be caused to regress, the particular therapeutic agent, and other clinical factors such as weight and condition of the subject and the route of administration of the therapeutic agent. It is to be understood that the methods, liquid formulations, and compositions described herein have application for both human and veterinary use, as well as uses in other possible animals. As described herein, tissue concentrations of therapeutic agents expressed in units of mass per volume generally refer to tissues that are primarily aqueous such as the vitreous, for example. Tissue concentrations of therapeutic agents expressed in unit of mass per mass generally refer to other tissues such as the sclera or retina tissues, for example. One concentration of BOL-303213-X that may be used in the methods described herein is one that provides about 10 ng/g to 300 ng/g or more of BOL-303213-X at the tissue level. Another concentration that may be used is one that provides about 100 ng/g to 200 ng/g or more at the tissue level. One of ordinary skill in the art would know how to arrive at an appropriate concentration depending on the route and duration of administration utilized, given the teachings herein.

Generally, the amount of BOL-303213-X administered in a liquid formulation is an amount sufficient to treat, prevent, inhibit, delay the onset, or cause regression of the disease or condition of the eye for the required amount of time. In some variations the amount of BOL-303213-X administered in the liquid formulation is an amount sufficient to treat the disease or condition of the eye for the required amount of time.

In some variations, any one or more of the formulations described herein are administered intravitreally every 3 or more months, every 6 or more months, every 9 or more months, or every 12 or more months, or longer, to treat one or more of choroidal neovascularization, wet AMD, dry AMD, to prevent wet AMD, or to prevent progression of dry AMD to wet AMD. In some variations, any one or more of the formulations described herein are administered subconjunctivally every 3 or more months, every 6 or more months, every 9 or more months, or every 12 or more months, or longer, to treat one or more of choroidal neovascularization, wet AMD, dry AMD, or to prevent wet AMD.

In some variations, any one or more of the BOL-303213-X formulations described herein are administered intravitreally every 3 or more months, every 6 or more months, every 9 or more months, or every 12 or more months, or longer, to treat one or more of choroidal neovascularization, wet AMD, dry AMD, to prevent wet AMD, or to prevent progression of dry AMD to wet AMD. In some variations, any one or more of the BOL-303213-X formulations described herein are administered subconjunctivally every 3 or more months, every 6 or more months, every 9 or more months, or every 12 or more months, or longer, to treat one or more of choroidal neovascularization, wet AMD, dry AMD, or to prevent wet AMD.

The liquid formulations, including but not limited to solutions, suspensions, emulsions and in situ gelling formulations, and compositions described herein may be used for delivery to the eye, as one nonlimiting example by ocular or periocular administration, of therapeutically effective amounts of BOL-303213-X for extended periods of time to treat, prevent, inhibit, delay the onset of, or cause regression of CNV, and thus may be used to treat, prevent, inhibit, delay the onset of, or cause regression of wet AMD, or transition of dry AMD to wet AMD. It is believed that by changing certain characteristics of the liquid formulations described herein, including but not limited to the components of the liquid formulations, the location in the eye to which the liquid formulation is delivered, including without limitation subconjunctival, sub-Tenon or intravitreal placement, the liquid formulations may be used to deliver therapeutically effective amounts of BOL-303213-X to the eye for a variety of extended time periods including delivery of therapeutic amounts for greater than about 1 week, for greater than about 2 weeks, for greater than about 3 weeks, for greater than about 1 month, for greater than about 3 months, for greater than about 6 months, for greater than about 9 months, for greater than about 12 months, for greater than about 18 months, or for greater than about 24 months.

When a therapeutically effective amount of BOL-303213-X is administered to a subject suffering from wet AMD, the BOL-303213-X may treat, inhibit, or cause regression of the wet AMD. Different therapeutically effective amounts may be required for treatment, inhibition or causing regression. A subject suffering from wet AMD may have CNV lesions, and it is believed that administration of a therapeutically effective amount of BOL-303213-X may have a variety of effects, including but not limited to causing regression of the CNV lesions, stabilizing the CNV lesion, and preventing progression of an active CNV lesion.

When a therapeutically effective amount of BOL-303213-X is administered to a subject suffering from dry AMD, it is believed that the BOL-303213-X may prevent or slow the progression of dry AMD to wet AMD.

The invention may be better understood by way of the following non-limiting examples.

### EXAMPLES

Abbreviations used herein are as follows:
Dio-Ac-LDL 3,3′-dioctadecyloxacarbocyanine perchlorate-acetylated-low density lipoprotein
IOP Intraocular pressure
rhVEGF Recombinant human vascular endothelial growth factor
ED₅₀ Concentration producing a 50% effect on the measured activity

Evaluation of Efficacy of BOL-303213-X In Human Retinal Endothelial Cells (HREC) Using the Network Formation Assay in Matrigel.

### Objective: This study was conducted to evaluate the ability of a novel tyrosine kinase inhibitor, BOL-303213-X, to prevent vascular endothelial cell network formation in an in vitro angiogenesis model—network formation assay in Matrigel.

Methods: Human retinal vascular endothelial cells were isolated and cultured from human donor eyes and characterized by Dio-Ac-LDL uptake. Cell network formation was evaluated after these cells were cultured in Matrigel in 1% FBS-RF6A medium with 20 ng/ml rhVEGF and various doses of BOL-303213-X (30, 60, 200, 600 and 1200 nM) for 24 hours. Cell isolates and cultures from three separated donors were used. The total cell network areas were computed by an image analysis software.

### Results: BOL-303213-X inhibited network formation of human retinal endothelial cells in Matrigel in the presence of VEGF showing an approximate ED₅₀ of 180 nM.

### Conclusions: BOL-303213-X was efficacious in inhibiting human retinal endothelial cell network formation in a dose dependent manner.

Evaluation of the In Vitro Efficacy and Cytotoxicity of BOL-303213-X In Endothelial Cells and in an Ex Vivo Model of Human choroid vascular growth.

Objective: The present study was conducted to evaluate the ability of a novel tyrosine kinase inhibitor, BOL-303213-X, to prevent vascular endothelial cell proliferation in vitro using human retinal, choroidal, and dermal microvascular endothelial cells, as well as bovine retinal endothelial cells. Inhibition of outgrowths in a human choroid explant assay was also used to evaluate BOL-303213-X activity in a more complex multi-cellular angiogenesis ex vivo model. Furthermore, caspase-3/7 and lactate dehydrogenase (LDH) activities were also determined following treatment of human retinal and choroidal endothelial cells with BOL-303213-X as a preliminary measure of cytotoxicity.
Methods: Human retinal and choroidal vascular endothelial cells were isolated and cultured from human donor eyes. Human dermal microvascular endothelial cells and bovine retinal endothelial cells were purchased from commercial sources. Vascular endothelial cell proliferation was measured by comparing the number of viable cells using a commercial cell viability assay kit. Inhibition of choroid outgrowths was evaluated in a human explant assay. Caspase-3/7 and LDH activities were utilized to assess cytotoxicity.
Results: BOL-303213-X inhibited proliferation of human retinal and choroidal endothelial cells and dermal microvascular endothelial cells, as well as bovine retinal endothelial cells, in a dose-dependent manner. BOL-303213-X was also effective in inhibiting choroid outgrowths in the human choroidal outgrowth assay. In confluent human retinal and choroidal endothelial cells, no significant increase in caspase-3/7 or LDH activity was observed over the range of concentrations used for the efficacy testing.

### Conclusions: BOL-303213-X demonstrates efficacy in both human and bovine ocular tissues in vitro, at concentrations below those associated with cytotoxicity.

In Vivo Ocular Efficacy and Tolerability Studies:

To expand the efficacy observation of BOL-303213-X in non-ocular system, an ocular efficacy study was conducted in rabbits that included ocular observations to evaluate tolerance of intravitreal administration of BOL-303213-X in hydroxypropyl methylcellulose (HPMC). A rabbit model of blood-retinal barrier (BRB) breakdown induced by intravitreal administration of recombinant human vascular endothelial growth factor (rhVEGF) was used. This model mimics pathologic situations such as macular edema, in which VEGF contributes to increased retinal vascular permeability resulting in edema.
In this study 30 μg BOL-303213-X in 50 μl of HPMC was given 3 or 5 days before the VEGF challenge with the evaluation of the BRB integrity also at 2 days after the VEGF challenge. BOL-303213-X given 3 days before the VEGF challenge significantly reduced the retinal vascular permeability induced by VEGF. There were no observed tolerance concerns except some eyes with blurred vitreous probably due to the suspension of the test article. Even though this efficacy study showed no dose-related, persistent tolerance concerns, an additional study was conducted in cats specifically to evaluate the tolerability of intravitreal 10 μg BOL-303213-X in HPMC and four pharmacokinetic studies with detailed ocular tolerability observations were also conducted. These studies confirmed that there were no major ocular tolerance concerns although certain studies showed visible aggregates of test articles in the vitreous after intravitreal injections given supratemporally.

BOL-303213-X/HPMC Suspension: Evaluation of Efficacy in Pigmented Rabbits Using a Model of Blood-Retinal Barrier Breakdown Induced by VEGF.

### Purpose: The aim of this study was to evaluate the efficacy of BOL-303213-X (30 μg) in hydroxypropyl methylcellulose (HPMC) suspension in pigmented rabbits using a model of blood-retinal barrier breakdown induced by rhVEGF 165.

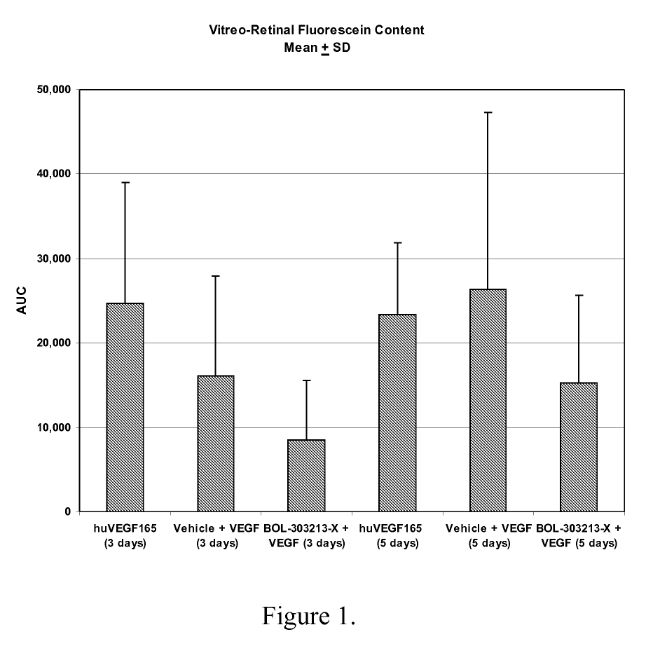

Methods: Eighty four (84) pigmented rabbits were allocated into 8 groups: three groups were given an intravitreal injection (50 μl) of BOL-303213-X (30 μg) in HPMC, three groups were given an intravitreal injection of HPMC vehicle and two groups were given no treatment. The rhVEGF 165 challenge was performed three days after the treatment injection to three groups (BOL-303213-X-treated, vehicle-treated, and untreated). The rhVEGF 165 challenge was also performed at five days after the treatment injection to four groups (BOL-303213-X-treated, vehicle-treated (n=2), and untreated). One group served as control for the tolerability of BOL-303213-X injection. Breakdown of the blood-retinal barrier was evaluated 24 h (one vehicle-treated group) and 48 h (the other six groups) after rhVEGF 165 challenge by fluorescein leakage from retinal vasculature into the vitreous using non-invasive scanning ocular fluorophotometry. Assessment of tolerability was evaluated by slit-lamp examination, laser flare measurements (LFM) of anterior chamber using a laser flare meter, intraocular pressure (IOP) measurements using a pneumotonometer and vitreo-retinal fluorophotometry. These examinations were performed before injection, before the rhVEGF165 challenge and the day of euthanasia.
Results: The general behavior of animals was not altered by the treatment. The body weights of the animals were within a normal range. No relevant complications were found during the clinical follow-up. There was no evidence of toxicity by slit-lamp examination, laser flare and IOP measurements after the intravitreal injection of BOL-303213-X or HPMC. In the experimental model of rhVEGF165-induced vascular leakage, three days after a single intravitreal injection of BOL-303213-X (30 μg) we found a statistically significant decrease (p=0.04; Mann and Whitney test for independent two-group comparison with p≦0.05 representing significance) in vitreous fluorescein leakage, compared to the rhVEGF 165 plus the HPMC vehicle-control group with a maximal inhibition of 52.7% (8,458.8±7,067.4 vs. 16,040.3±11,872.8 (mean±SD, n=12 per group) (FIG. 1). There was a trend towards inhibition at 5 days (57.7% inhibition), but differences between vehicle and BOL-303213-X treated eyes (15,228.2±10,364.6 vs 26,382.6±20,926.4 (mean±SD, n=12 per group) were not statistically significant (p>0.05).
Conclusion: 30 μg of BOL-303213-X in HPMC suspension was effective at blocking experimental VEGF-induced blood-retinal barrier breakdown when it was injected 3 days before the rhVEGF165 challenge. BOL-303213-X in HPMC suspension at 30 μg-dose appeared to be safe in normal pigmented rabbits.

BOL-303213-X/HPMC Suspension: Evaluation of Efficacy in Monkeys Using a Laser-Induced Choroidal Neovasuclarization Model.

Purpose: This study was done to qualitatively evaluate the potential inhibitory effect of BOL-303213-X on laser-induced choroidal neovascularization in cynomolgus monkeys when administered biweekly by intravitreal injection beginning at the time of laser treatment.

Methods: Eight cynomolgus monkeys (Macaca fascicularis) were used in this study. The animals were assigned to the study using a computerized procedure designed to achieve body weight balance with respect to groups. The animals were anesthetized with ketamine and xylazine and the eyes dilated with a mydriatic. The macula of both eyes of each animal received laser photocoagulation treatment using a 532 nm diode green laser and slit lamp delivery system and a plano fundus contact lens. This is a standard method for inducing choroidal neovascularization. Nine areas were symmetrically placed in the macula of each eye. The laser parameters included a 75 micron spot size, 0.1 second duration at power settings ranging from 400 to 750 mW. Following laser treatment, BOL-303213-X in HPMC (45 μg) was injected into the vitreous of one eye of each animal and the HPMC-vehicle control article was injected into the contralateral eye. A second injection of BOL-303213-X in HPMC (45 μg) to the treated eye and of the HPMC-vehicle to the control eye was made two weeks post-laser. Fluorescein angiography was performed on both eyes of all animals before dosing and approximately 2, 3, and 4 weeks post-laser. Fuorescein angiograms were evaluated according to the following grading system for evidence of excessive permeability (i.e. fluorescein leakage). Grade IV lesions are considered to indicate presence of choroidal neovascularization.

| Lesion | |
|---|---|
| Grade | Definition |
| I | No hyperfluorescence |
| II | Hyperfluorescence without leakage |
| III | Hyperfluorescence early or mid-transit and late leakage |
| IV | Bright hyperfluorescence early or mid-transit and late |
| | leakage beyond borders of treated area |

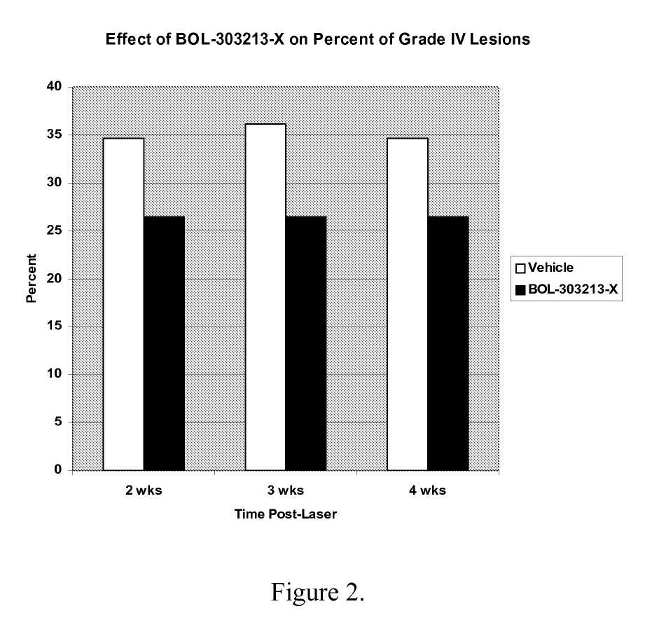

### Results: At 2, 3 and 4 weeks post-laser the percent of grade IV lesions was consistently lower in the eyes treated with BOL-303213-X (FIG. 2).

### Conclusion: 45 μg of BOL-303213-X in HPMC suspension was effective at inhibiting choroidal neovascularization when it was injected after laser challenge.

BOL-303213-X In HPMC Suspension. Evaluation of Ocular Tolerability Following a Single Intravitreal Administration in Pigmented Rabbits.

### Purpose: The aims of this study were to determine the ocular tolerability and pharmacokinetics of BOL-303213-X in Hydroxy Propyl Methyl Cellulose (HPMC) suspension after a single intravitreal injection at 4 doses: 10, 15, 20 and 30 μg.

Methods: One hundred and two rabbits were allocated into four-dose groups (group 1: 10 μg, group 2: 15 μg, group 3: 20 μg and group 4: 30 μg of BOL-303213-X in HPMC suspension) and one control group (HPMC). Each group was divided in four time-points (15 min, Days 1, 3 and 5) except the control group (one time-point: Day 5). Each animal received an intravitreal injection of test article (50 μl) in the right eye. Tolerability was evaluated by slit lamp examination, anterior chamber inflammation (flare) by laser flare measurements (LFM), intraocular pressure measurements and integrity of the blood retinal barrier (BRB) by vitreous fluorophotometry. These examinations were performed before injection and, on Days 1, 3 and 5.
Results: The general behavior of animals was not altered by the treatment. The body weights of the animals were within a normal range. No relevant complications were found during the clinical follow-up. There was no evidence of toxicity by clinical examination, laser flare and IOP measurements after the BOL-303213-X or HPMC intravitreal injection. Following the intravitreal injection, a small aggregate of drug precipitates was clinically observed in the vitreous. At the end of the study period (Day 5), fundus examination revealed some inflammatory cells in the vitreous cavity in 9 rabbits out of 30, at least 1 animal of each group. Some mild blurring vitreous was observed but the fluorescein leakage evaluation did not show any observable retinal change. The blood-retinal barrier was not affected by the different injections. A transient anterior inflammation was observed 1 day after the injection but this was not dose-dependent. There was no difference in IOP between the BOL-303213-X injected and control eyes at any time points.
Conclusion: Based on ophthalmic data, BOL-303213-X in HPMC suspension at 10, 15, 20 and 30 μg-doses appeared to be safe in normal pigmented rabbit.

What is claimed is:

1. A formulation comprising a therapeutic agent, wherein the formulation when placed into the vitreous of a rabbit eye delivers an amount of the therapeutic agent sufficient to achieve, for a period of time of at least 30 days following administration of the liquid formulation, an average concentration of therapeutic agent in the retina tissues of the rabbit eye equivalent to a BOL-303213-X concentration of at least 10 ng/g retina tissue.

2. A formulation comprising a therapeutic agent, wherein the liquid formulation when placed into the vitreous of a rabbit eye delivers an amount of the therapeutic agent sufficient to achieve, for a period of time of at least 30 days following administration of the formulation, an average concentration of therapeutic agent in the retina choroid tissue of the rabbit eye equivalent to a BOL-303213-X concentration of at least 300 ng/g retina tissue.

3. The formulation of claim 1 wherein the formulation is a liquid.

4. The formulation of claim 1 wherein the formulation is a solid.

5. The formulation of claim 1 wherein the formulation is biodegradable.

6. The formulation of claim 1 wherein the formulation is non-biodegradable.

7. A method of treating an angiogenesis-mediated disease of the eye; the method comprising administering to a patient afflicted with an angiogenesis-mediated disease of the eye a therapeutically effective amount of the formulation of claim 1.

8. A method of treating an inflammatory-mediated disease of the eye, the method comprising administering to a patient afflicted with an inflammatory-mediated disease of the eye a therapeutically effective amount of the formulation of claim 1.

9. The method of claim 7 wherein the angiogenesis-mediated disease of the eye is selected from the group consisting of melanoma, alkali burns, pteragtium, herpetic stromal keratitis, trachoma, proliferative diabetic retinopathy, retinopathy of prematurity, choroidal neovascularization, retinal neovascularization and glaucoma.

10. The method of claim 8 wherein the inflammatory-mediated disease of the eye is selected from the group consisting of diabetic edema, macular edema, dry eye, allergic conjunctivitis, retinal degeneration and glaucoma.

11. The formulation of claim 3, wherein the liquid formulation forms a non-dispersed mass when injected into the vitreous of a rabbit eye.

12. The formulation of claim 3, wherein the liquid formulation forms a dispersed mass when injected into the vitreous of a rabbit eye.

13. A method for treating wet age-related macular degeneration in a human subject, the method comprising administering to the human subject by intraocular or periocular delivery a volume of a liquid formulation containing an amount of BOL-303213-X effective to treat wet age-related macular degeneration in the human subject.

14. The method of claim 13, wherein the liquid formulation further comprises hydroxy propyl methylcellulose and wherein the volume of liquid formulation is administered to the human subject by placement in the vitreous and the volume of liquid formulation contains less than 100 μl of hydroxy propyl methyl cellulose.

15. The method of claim 13, wherein the liquid formulation further comprises hydroxy propyl methyl cellulose and wherein the volume of liquid formulation is administered to the human subject by sub-Tenon placement between the sclera and conjunctiva and the volume of liquid formulation contains less than 150 μl of hydroxy propyl methyl cellulose.

16. A method for treating wet age-related macular degeneration in a human subject, the method comprising administering to the human subject by intraocular or periocular delivery of a liquid formulation comprising an effective amount of BOL-303213-X and pharmaceutically acceptable salts and esters thereof and wherein the liquid formulation has one or more characteristics selected from the group consisting of (1) the liquid formulation when injected into the vitreous of a rabbit eye delivers an amount of the therapeutic agent sufficient to achieve, for a period of time of at least 30 days following administration of the liquid formulation, an average concentration of therapeutic agent in the retina choroid tissues of the rabbit eye equivalent to a BOL-303213-X concentration of at least 10 ng/g retina tissue; (2) the liquid formulation when injected into the vitreous of a rabbit eye delivers an amount of the therapeutic agent sufficient to achieve, for a period of time of at least 30 days following administration of the liquid formulation, an average concentration of therapeutic agent in the vitreous of the rabbit eye equivalent to a BOL-303213-X concentration of at least 300 ng/g retina tissue; (3) the liquid formulation when sub-Tenon injected between the Tenon's capsule and conjunctiva of a rabbit eye delivers an amount of the therapeutic agent sufficient to achieve, for a period of time of at least 30 days following administration of the liquid formulation, an average concentration of therapeutic agent in the retina choroid tissue of the rabbit eye equivalent to a BOL-303213-X concentration of at least 10 ng/mg retina tissue; (4) the liquid formulation when injected between the sclera and conjunctiva of a rabbit eye delivers an amount of the therapeutic agent sufficient to achieve, for a period of time of at least 30 days following administration of the liquid formulation, an average concentration of therapeutic agent in the retina tissues of the rabbit eye equivalent to a BOL-303213-X concentration of at least 300 ng/mg retina tissue; and (5) the liquid formulation forms a dispersed mass when injected into the vitreous of a rabbit eye.
